(19) United States
(12) Patent Application Publication
(10) Pub. No.: US 2015/0226685 A1
(43) Pub. Date: Aug. 13, 2015

(54) SYSTEMS AND METHODS FOR QUANTIFYING MULTIPLE REFRACTIONS WITH DIFFRACTION ENHANCED IMAGING

(71) Applicant: MUSC Foundation for Research Development, Charleston, SC (US)

(72) Inventor: Dean M. Connor, Mount Pleasant, SC (US)

(21) Appl. No.: 14/620,680

(22) Filed: Feb. 12, 2015

Related U.S. Application Data
(60) Provisional application No. 61/939,087, filed on Feb. 12, 2014.

(51) Int. Cl. G01N 23/207; A61B 6/00

(52) U.S. Cl. CPC G01N 23/207; A61B 6/484

(57) ABSTRACT

Systems and methods for detecting small angular changes in an X-ray beam caused by multiple refractions within an object. According to an aspect, a method for detecting an image of an object includes providing a single X-ray source. The method also includes generating a first X-ray beam. Further, the method includes positioning monochromator crystals to intercept the first X-ray beam such that second X-ray beams are produced. The method also includes positioning an object in paths of the second X-ray beams for transmission of the second X-ray beams through the object and emitting from the object transmitted X-ray beams. The method also includes directing the transmitted X-ray beams at angles of incidence on analyzer crystals, wherein the angles of incidence of the analyzer crystals are independently adjustable. Further the method includes detecting an image of the object from each of the X-ray beams diffracted from the analyzer crystals.

### CROSS REFERENCE TO RELATED APPLICATION

This application claims priority to and the benefit of U.S. Provisional Patent Application No. 61/939,087, filed Feb. 12, 2014 and titled SYSTEMS AND METHODS FOR QUANTIFYING MULTIPLE REFRACTIONS WITH DIFFRACTION ENHANCED IMAGING; the content of which is incorporated herein by reference in its entirety.

### TECHNICAL FIELD

The subject matter disclosed herein relates to X-ray imaging. More particularly, the subject matter disclosed herein relates to systems and methods for detecting small angular changes in an X-ray beam caused by multiple refractions within an object by use of X-ray beams and selective angular alignment of the X-ray optics.

### BACKGROUND

X-ray imaging has been used in a variety of fields for imaging objects. For example, X-ray imaging has been used extensively in the medical field for non-destructive testing and X-ray computed tomography (CT). Various other types of technology are also being used for medical imaging. For example, diffraction enhanced imaging (DEI) is an X-ray imaging technique that extends the capability of conventional X-ray imaging.

Diffraction enhanced imaging (DEI) is a phase contrast x-ray imaging modality. As with other phase contrast X-ray imaging modalities, DEI's image contrast is, in part, from the refraction of X-rays. In contrast, conventional X-ray imaging techniques measure only X-ray attenuation. DEI utilizes perfect crystal diffraction to convert small angular changes in the X-ray beam, caused by interactions within the imaging field, into large intensity changes in the final image.

The use of a silicon analyzer crystal in the path of the X-ray beam generates additional image contrast from X-ray refraction. DEI utilizes highly collimated X-rays prepared by X-ray diffraction from perfect single-crystal silicon. These collimated X-rays are of single X-ray energy, practically monochromatic, and are used as the beam to image an object.

Objects that have very little absorption contrast may have considerable refraction contrast, thus improving visualization and extending the utility of X-ray imaging. Applications of DEI techniques to biology and materials science have generated significant gains in both contrast and resolution, indicating the potential for use in mainstream medical imaging. An area of medicine where DEI may be particularly effective is in breast imaging for cancer diagnosis, where the diagnostic structures of interest often have low absorption contrast, making them difficult to see. Structures with low absorption contrast, such as the speculations extending from a malignant mass, have high refraction and ultra-small angle scatter contrast. It is desirable to provide a DEI system with the capability to increase both the sensitivity and specificity of X-ray-based breast imaging.

Multiple studies have demonstrated improved image contrast in both medical and industrial applications of DEI. Advantages of DEI systems over conventional X-ray imaging systems in the medical field include a dramatic reduction in patient radiation dose and improved image quality. The dose reduction is due to the ability of DEI systems to function at higher X-ray energies. X-ray absorption is governed by the photoelectric effect, Z²/E³, where Z is the atomic number and E is the photon energy.

A monoenergetic radiograph contains several components that can affect image contrast and resolution: a coherently scattered component I_{C}, an incoherently scattered component I_{I}, and a transmitted component. X-rays passing through an object or medium where there are variations in density can be refracted, resulting in an angular deviation. Specifically, deviations in the X-ray range result from variations in pt along the path of the beam, where ρ is the density and t is the thickness. A fraction of the incident photons may also be diffracted by structures within an object, which are generally on the order of milliradians and referred to as small angle scattering. The sum total of these interactions contributed to the recorded intensity in a radiograph I_{N}, which can be represented by the following equation:

I_{N}=I_{R}+I_{D}+I_{C}+I_{I}

System spatial resolution and contrast can be degraded by the contributions of both coherent and incoherent scatter. Anti-scatter grids are often used in medical imaging to reduce the contribution of scatter, but their performance is limited and use of a grid often requires a higher dose to compensate for the loss in intensity.

The DEI technique may utilize a silicon analyzer crystal in the path of the post-object X-ray beam to virtually eliminate the effects of both coherent and incoherent scatter. The narrow angular acceptance window of the silicon analyzer crystal may be referred to as its rocking curve or reflectivity profile, and is on the order of microradians for the X-ray energies used in DEI. The analyzer acts as an exquisitely sensitive angular filter, which can be used to measure both refraction and extinction contrast. Extinction contrast is defined as the loss of intensity from the incident beam due to scattering, which can produce substantial improvements in both contrast and resolution.

The Darwin Width (DW) is used to describe reflectivity curves, and is approximately the Full Width at Half Maximum (FWHM) of the reflectivity curve. Points at −½ DW and +½ DW are points on the curve with a steep slope, producing the greatest change in photon intensity per microradian for a particular analyzer reflection and beam energy. Contrast at the peak of the analyzer crystal rocking curve is dominated by X-ray absorption and multiple refraction (sometimes referred to as extinction), resulting in near scatter-free radiographs. Refraction contrast is highest where the slope of the rocking curve is greatest, at the −½ and +½ DW positions. One DEI based image processing technique uses these points to extract the contrast components of refraction and apparent absorption from these image pairs.

The following paragraph describes of this technique for extracting the contrast components of refraction and apparent absorption from an image pair. When the analyzer crystal is set to an angle representing +/−½ DW for a given reflection and beam energy, the slope of the rocking curve is relatively consistent and can be represented as a two-term Taylor series approximation as represented by the following equation:

$$R ( {θ}_{0} + {Δθ}_{z} ) = R ( {θ}_{0} ) + \frac{d R}{d θ} ( {θ}_{0} ) {Δθ}_{z} .$$

If the analyzer crystal is set to the low-angle side of the rocking curve (−½ DW), the resulting image intensity can be represented by the following equation:

$${I}_{L} = {I}_{R} ( R ( {θ}_{L} ) + \frac{d R}{d θ} {\vert}_{θ = {θ}_{L}} {Δθ}_{z} ) .$$

The recorded intensity for images acquired with the analyzer crystal set to the high-angle position (+½ DW) can be represented by the following equation:

$${I}_{H} = {I}_{R} ( R ( {θ}_{H} ) + \frac{d R}{d θ} ( {θ}_{H} ) {Δθ}_{z} ) .$$

These equations can be solved for the changes in intensity due to apparent absorption (IR) and the refraction in angle observed in the z direction (ΔθZ) represented by the following equation:

$${Δθ}_{z} = \frac{{I}_{H} R ( {θ}_{L} ) - {I}_{L} R ( {θ}_{H} )}{{I}_{L} ( \frac{d R}{d θ} ) ( {θ}_{H} ) - {I}_{H} ( \frac{d R}{d θ} ) ( {θ}_{L} )}$$

$${I}_{R} = \frac{{I}_{L} ( \frac{d R}{d θ} ) ( {θ}_{H} ) - {I}_{H} ( \frac{d R}{d θ} ) ( {θ}_{L} )}{R ( {θ}_{L} ) ( \frac{d R}{d θ} ) ( {θ}_{H} ) - R ( {θ}_{H} ) ( \frac{d R}{d θ} ) ( {θ}_{L} )} .$$

These equations can be applied to the high and low angle images on a pixel-by-pixel basis to separate the two contrast elements into what is known as a DEI apparent absorption and refraction image. However, it is important to note that each of the single point rocking curve images used to generate DEI apparent absorption and refraction images is useful.

Development of a clinical DEI imager may have significance for women's health and medical imaging in general for the following reasons: (1) DEI has been shown to produce very high contrast for the features that are most important to detection and characterization of breast cancer; (2) the physics of DEI allows for imaging at higher x-ray energies than used with absorption alone; and (3) the ability of DEI to generate contrast without the need of photons to be absorbed dramatically reduces ionization, and thus reduces the absorbed dose.

Current DEI and DEI imaging processing techniques are based heavily on conventional imaging theory and rely, at least in part, on X-ray absorption for image generation. Thus, objects imaged using these techniques absorb radiation. Such radiation exposure is undesirable in applications for medical imaging given concerns of dose, and this reasoning places considerable engineering limitations that make clinical and industrial translation challenging. Thus, it is desirable to provide DEI and DEI techniques that produce high quality images and that rely less on absorption but produce images with improved, or at least equivalent, diagnostic quality and feature visualization. In addition, it is desirable to reduce DEI imaging time, which can be affected by the significant reduction of beam flux in DEI monochromators.

Accordingly, in view of desired improvements associated with DEI and DEI systems, there exists a need for improved DEI and DEI systems and related methods for detecting an image of an object.

### SUMMARY

Systems and methods for detecting a small angular changes in an x-ray beam caused by multiple refractions within an object by use of X-ray beams and selective angular alignment of the x-ray optics.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

According to one embodiment, a method for detecting an image of an object includes providing a single X-ray source. The method further includes generating a first X-ray beam using the single X-ray source. The method also includes positioning a plurality of monochromator crystals to intercept the first X-ray beam such that a plurality of second X-ray beams each having predetermined energy level, is produced. The method further includes positioning an object in paths of the plurality of second X-ray beams for transmission of the plurality of second X-ray beams through the object and emitting from the object a plurality of transmitted X-ray beams. The method further includes directing the plurality of transmitted X-ray beams at angles of incidence upon a plurality of analyzer crystals, wherein the angles of incidence of the analyzer crystals are independently adjustable. The method further includes detecting an image of the object from each of the X-ray beams diffracted from each analyzer crystal using a plurality of detectors.

According to another embodiment, the plurality of detectors comprise at least one high spatial resolution detector and at least one low spatial resolution detector.

According to another embodiment, the low spatial resolution detector is an energy-resolving detector.

According to another embodiment, further including adjusting the angles of incidence of the analyzer crystals, and detecting a plurality of images of the object during adjustment of the angles of incidence of the analyzer crystals.

According to another embodiment, directing the plurality of transmitted X-ray beams comprises directing the rotation of the analyzer crystals about a propagation direction of the transmitted X-ray beams relative to the analyzer crystal.

According to another embodiment, detecting an image of the object comprises tilting the analyzer crystal out of alignment by a predetermined chi-angle; and the method further includes detecting a plurality of images of the object in sequence for a range of theta-angular positions of the analyzer crystals.

According to another embodiment, further including using the detectors to measure the intensity of the diffracted X-ray beam.

According to another embodiment, further including using the measured intensity of the diffracted X-ray beam to determine the degree of anisotropy in a structure of the object.

According to another embodiment, further including using the measured intensity of the diffracted X-ray beam to determine the orientation direction of structures in the object.

According to another embodiment, measuring the intensity of the diffracted X-ray beam comprises detecting a plurality of intensity measurements for a range of angular positions of the analyzer crystal.

According to another embodiment, the range of angular positions is a range of angles the X-ray source is rotated about the propagation direction of the X-ray beam relative to the analyzer crystals.

According to another embodiment, the range of angular positions is a range of angles the object is rotated about the propagation direction of the X-ray beam relative to the analyzer crystals.

According to another embodiment, further including using the series of intensity measurements to determine the degree of anisotropy in the structure of the object.

According to another embodiment, further including using the series of intensity measurements to determine the orientation direction of the structures in the object.

According to another embodiment, measuring the intensity of the diffracted X-ray beam comprises tilting the crystal analyzer out of alignment by a predetermined angle; and wherein the method further comprises detecting a series of intensity measurements are obtained for a range of angular positions of the analyzer crystal.

According to another embodiment, further including using the plurality of intensity measurements to determine the maximum and minimum reflectivity profile widths.

According to another embodiment, a system for detecting an image of an object includes a single X-ray source configured to generate a first X-ray beam. The system also includes a plurality of monochromator crystals positioned to intercept the first X-ray beam such that a plurality of second X-ray beams each having predetermined energy level, is produced. The system also includes a plurality of analyzer crystals positioned to intercept a plurality of transmitted X-ray beams at an angle of incidence from the object, wherein the plurality of transmitted X-ray beams are emitted from the object positioned in the path of the plurality of second X-ray beams, and wherein the angles of incidence of the analyzer crystals are independently adjustable. The system also includes a plurality of detectors configured to detecting an image of the object from each of the transmitted X-ray beams diffracted from each analyzer crystal.

According to another embodiment, the plurality of detectors comprise at least one high spatial resolution detector and at least one low spatial resolution detector.

According to another embodiment, the low spatial resolution detector is an energy-resolving detector.

According to another embodiment, further includes the plurality of detectors configured to adjust the angles of incidence of the analyzer crystals, and detect a plurality of images of the object during adjustment of the angles of incidence of the analyzer crystals.

According to another embodiment, the analyzer crystals are configured to rotate about a propagation direction of the transmitted X-ray beams relative to the analyzer crystal for directing the angles of incidence of the plurality of transmitted X-ray beams.

According to another embodiment, the system is configured to tilt the analyzer crystal out of alignment by a predetermined chi-angle; and the detectors are further configured to detect a plurality of images of the object in sequence for a range of theta-angular positions of the analyzer crystals.

According to another embodiment, the detectors are further configured to measure the intensity of the diffracted X-ray beam.

According to another embodiment, the detectors are further configured to determine the degree of anisotropy in a structure of the object based on the measured intensity of the diffracted X-ray beam.

According to another embodiment, the detectors are further configured to determine the orientation direction of a structure in the object based on the measured intensity of the diffracted X-ray beam.

According to another embodiment, measuring the intensity of the diffracted X-ray beam comprises detecting a plurality of intensity measurements for a range of angular positions of the analyzer crystal.

According to another embodiment, the range of angular positions is a range of angles the X-ray source is rotated about the propagation direction of the X-ray beam relative to the analyzer crystals.

According to another embodiment, the range of angular positions is a range of angles the object is rotated about the propagation direction of the X-ray beam relative to the analyzer crystals.

According to another embodiment, the detectors are further configured to determine the degree of anisotropy in a structure based on the plurality of intensity measurements.

According to another embodiment, the detectors are further configured to determine the orientation direction of the structure in the object based on the plurality of intensity measurements.

According to another embodiment, measuring the intensity of the diffracted X-ray beam comprises the analyzer crystals further configured to be tilted out of alignment by a predetermined angle; and the detectors are further configured to detect a plurality of intensity measurements for a range of angular positions of the analyzer crystals.

According to another embodiment, the detectors are further configured to determine the maximum and minimum reflectivity profile widths using the plurality of intensity measurements.

### BRIEF DESCRIPTION OF THE DRAWINGS

The foregoing summary, as well as the following detailed description of various embodiments, is better understood when read in conjunction with the drawings provided herein. For the purposes of illustration, there is shown in the drawings exemplary embodiments; however, the presently disclosed subject matter is not limited to the specific methods and instrumentalities disclosed. In the drawings:

FIG. 1 is a block diagram of an example of a top and side view diffraction enhanced imaging (DEI) system for quantifying multiple refractions with diffraction enhanced imaging, according to an embodiment of the subject matter described herein;

FIG. 2 is a graph of an example of X-ray beam intensity as a function of theta-angle relative to a Bragg diffraction peak, according to an embodiment of the subject matter described herein;

FIG. 3 is a diagram showing example definitions of the angles theta and chi on an analyzer crystal, according to an embodiment of the subject matter described herein;

FIG. 4 is a graph showing example maximum angular width and minimum angular width of DEI reflectivity profiles using a bovine cortical bone sample, according to an embodiment of the subject matter described herein;

FIG. 5 is a diagram showing exemplary X-ray beams passing through multiple refracting structures and the effect of multiple refractions on the measured reflectivity profile width, according to an embodiment of the subject matter described herein;

FIG. 6 is a diagram showing exemplary reflectivity profiles for a chi-aligned and chi-misaligned X-ray beam, according to an embodiment of the subject matter described herein;

FIGS. 7A and 7B are diagrams showing exemplary effects on the reflectivity profiles with misalignment of the χ-angle of the analyzer crystal by a predetermined amount, according to an embodiment of the subject matter described herein;

FIG. 8 is a diagram showing the exemplary effect shape and angular orientation of structures may affect the X-ray beam, according to an embodiment of the subject matter described herein;

FIGS. 9-19 are schematic diagrams of different example DEI systems including multiple monochromator crystals and multiple small area sources according to embodiments of the subject matter described herein;

FIG. 20 is a flow chart of an exemplary process for imaging an object by use of a DEI system, such as one of the DEI systems shown in FIGS. 9-19, according to an embodiment of the subject matter described herein;

FIG. 21 is a schematic diagram of an example DEI system that can utilize facing sides of adjacent monochromator crystals for detecting an image of an object according to embodiments of the subject matter described herein;

FIG. 22 is a flow chart of an exemplary process for imaging an object by use of a DEI system, such as one of the DEI system shown in FIG. 21, according to embodiments of the subject matter described herein;

FIG. 23 is a side view of an example analyzer crystal of any one of the DEI systems shown in FIGS. 9-19 and 21 according to embodiments of the subject matter described herein.

FIG. 24 is a flow chart of an exemplary process for the simultaneous acquisition of high spatial resolution images and low spatial resolution, and high angular resolution images of an object by use of a DEI system, according to embodiments of the subject matter described herein;

FIG. 25 is a flow chart of an exemplary process for the acquisition of high angular resolution images at a range of object orientations with respect to a DEI system, according to embodiments of the subject matter described herein;

FIGS. 26A and 26B depict block diagrams of examples of a top and side view of DEI systems, where the object or the DEI system are rotated relative to the other, according to embodiments of the subject matter described herein;

FIG. 27 is a flow chart of an exemplary process for the simultaneous acquisition of high angular resolution images at multiple X-ray energies with a DEI optical system, according to embodiments of the subject matter described herein; and

FIG. 28 is a flow chart of an exemplary process for the acquisition of high angular resolution images at multiple X-ray energies with a DEI optical system with the chi angle of the analyzer crystals shifted by a predetermined amount from the aligned angular position, according to embodiments of the subject matter described herein.

### DETAILED DESCRIPTION

The presently disclosed subject matter is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies. Moreover, although the term “step” may be used herein to connote different aspects of methods employed, the term should not be interpreted as implying any particular order among or between various steps herein disclosed unless and except when the order of individual steps is explicitly described.

The subject matter described herein discloses improved diffraction enhanced imaging (DEI) and DEI systems and related methods for detecting images with multiple refractions from an object.

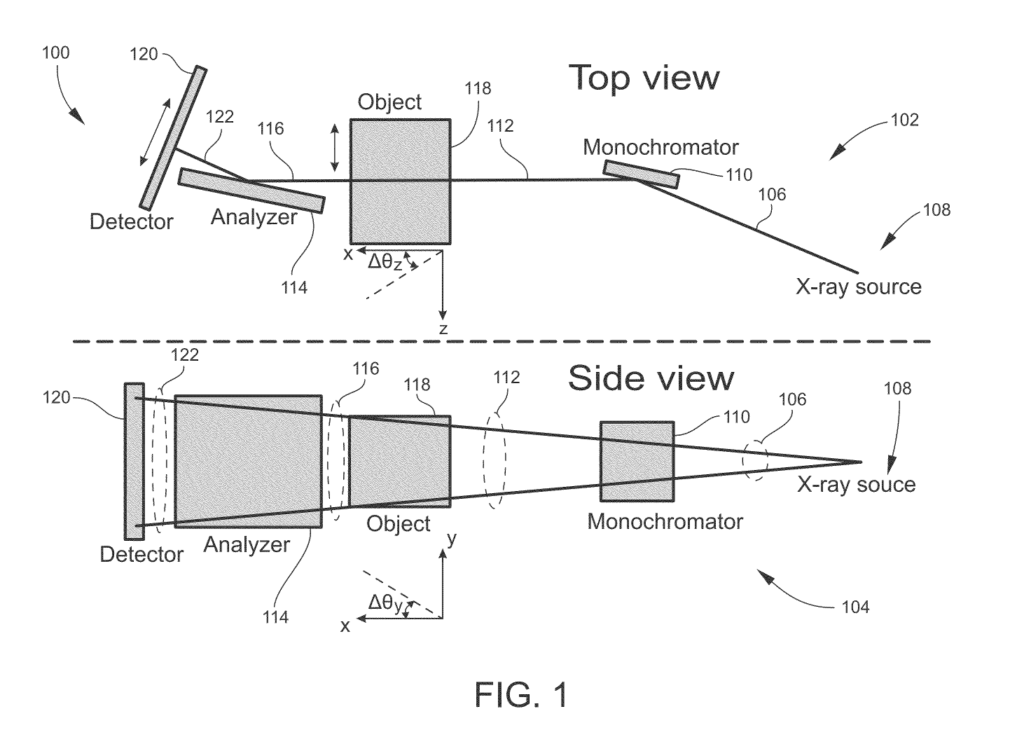

Referring to FIG. 1, a block diagram of a DEI system 100 including a top view 102 and a side view 104 is illustrated, according to embodiments described herein. An X-ray beam 106 from an X-ray source 108 is incident upon one or more monochromator crystals 110 that may be set to an angle to diffract the desired energy within the polyenergetic X-ray beam 106, according to Bragg's Law of diffraction. If a divergent X-ray source is used (e.g. an X-ray tube source), then the divergence in one direction is suppressed (up/down in the top view 102), but the divergence in the perpendicular direction (up/down in the side view 104) is maintained. One or more monochromator crystals 110 may be positioned to intercept the X-ray beam 106 such that a plurality of second X-ray beams 112, each having a predetermined energy level, may be produced. One or more analyzer crystals 114 may be positioned to intercept one or more transmitted X-ray beams 116 at an angle of incidence from an object 118. The one or more transmitted X-ray beams 116 may be emitted from the object 118, where the object 118 is positioned in the path of the plurality of second X-ray beams 112. Additionally, the angles of incidence of the analyzer crystals 114 may be independently adjustable. One or more detectors 120 may be configured to detect an image of the object 118 from each of the transmitted X-ray beams 116 diffracted 122 from each analyzer crystal 114.

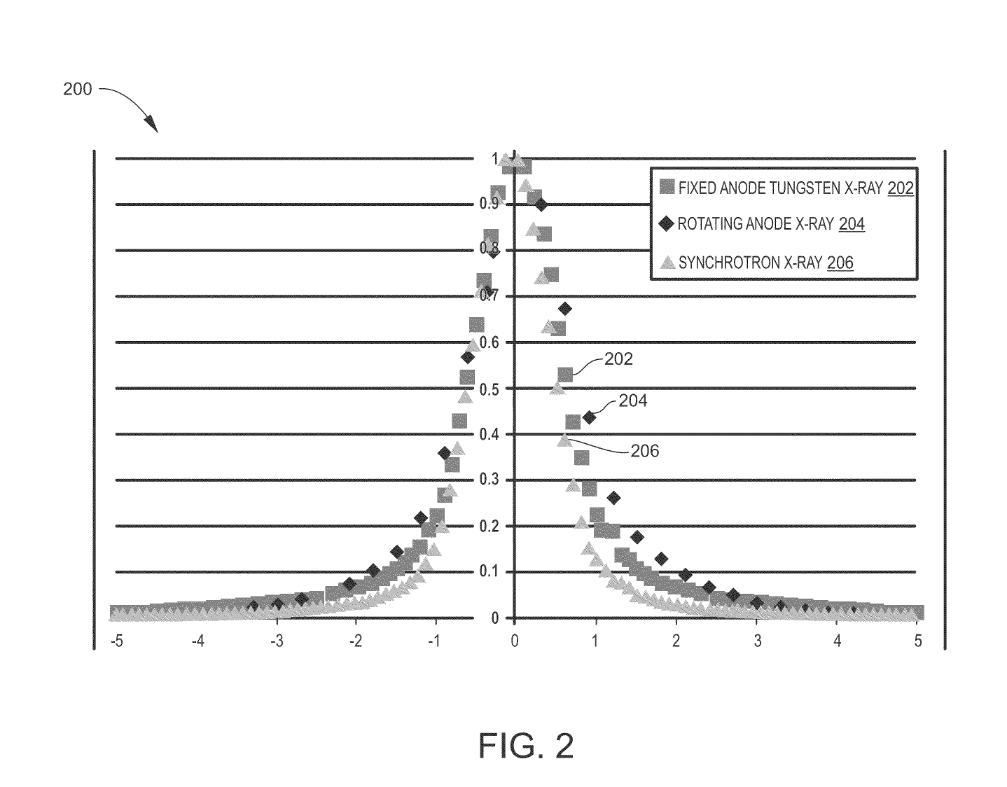

Referring to FIG. 2, the DEI system's 100 analyzer crystal 114 has an intrinsic reflectivity profile 200, R. The reflectivity profile 200 is a measure of the ratio of the intensity of the diffracted X-ray beam 122 to the incident X-ray beam 116 on the analyzer crystal 114. The diffracted X-ray beam 122 is a function of the angle of the transmitted X-ray beam 116 to the plane of the analyzer crystal 114 face. The intrinsic angular width of the reflectivity profile 200 may be a function of the analyzer crystal 114 type used for the optics (for example, silicon or germanium), the number of analyzer crystals 114 in the system 100, the diffraction plane, and the energy of the X-ray beam. For a system with one silicon monochromator and one silicon analyzer crystal, and using the [333] diffraction plane and a relevant range of imaging energies (10-100 keV), the full-width at half-maximum of the intrinsic reflectivity profile, Wᵢₙₜ, is in the range of about 1-10 microradians. The intrinsic reflectivity profile is an example of a reflectivity profile with no object in the imaging field.

In the example illustrated by FIG. 2, intensity (y-axis) is shown as a function of theta-angle (x-axis; in micoradians) relative to the Bragg diffraction peak. Measured with a fixed anode tungsten X-ray tube source 202, a rotating anode X-ray tube source 204, and a synchrotron X-ray source 206 for an X-ray energy of about 60 keV and the silicon [333] diffraction peak.

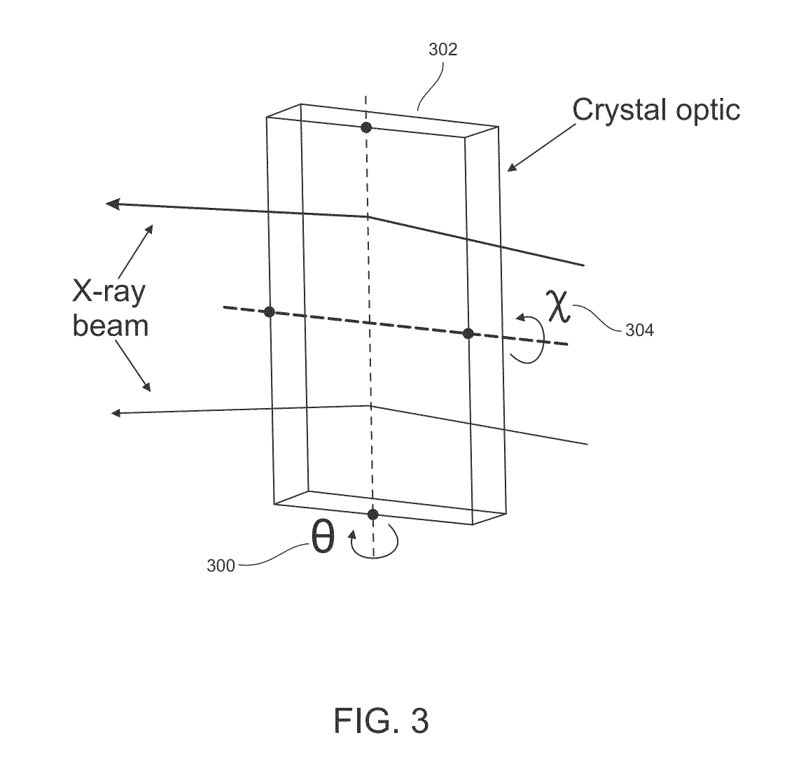

Referring now to FIG. 3, the intrinsic reflectivity profile 200 is taken by changing the angle, θ 300 (theta) of an analyzer crystal 302. The angle the DEI beam makes with respect to the diffraction plane is changed directly as:

φ′−φ=Δφ=Δθ Equation 1

Where φ is the Bragg angle for the diffraction peak, φ′ is the new angle with respect to diffraction plane, and Δθ is the change in the θ 300 angle with respect to the Bragg diffraction peak. Thus, DEI is most sensitive to changes in this the angle, θ 300. Conversely, an intrinsic reflectivity profile taken by changing the angle χ 304 of the analyzer crystal 302 could be much wider, since tilting the diffraction plane by the angle χ 304, only changes the angle of the X-ray beam with respect to the diffraction plane by a small amount denoted in Equation 2,

Δφ=φ′−φ=sin⁻¹(sin Ø cos Δχ)−φ Equation 2

where φ and φ′ are as described above and Δχ is the change in the χ 304 angle with respect to the Bragg diffraction peak. Small changes in φ, will require significantly larger changes in χ 304.

As an example, when an object is placed in the path of the X-ray beam, the object may refract the X-ray beam. The change in angle (resulting from the refraction of the beam) can be broken down into its x- and z-components (FIGS. 1A and 1B). A change in angle, Δθ_{z} (an upward/downward deflection in FIG. 1A), is equivalent to shifting the θ 300 angle of the analyzer crystal, and the reflectivity at the new point is R(θ+Δθ_{z}). A change in angle, Δθₓ (an upward/downward deflection in FIG. 1B), is equivalent to shifting the χ 304 angle of the analyzer crystal. Therefore, DEI's optics may be more sensitive to changes in the angle Δθ_{z} of the X-ray beam than they are to changes in the θ-angle in the orthogonal plane, Δθₓ. This difference in angular sensitivity is a desired aspect for this invention.

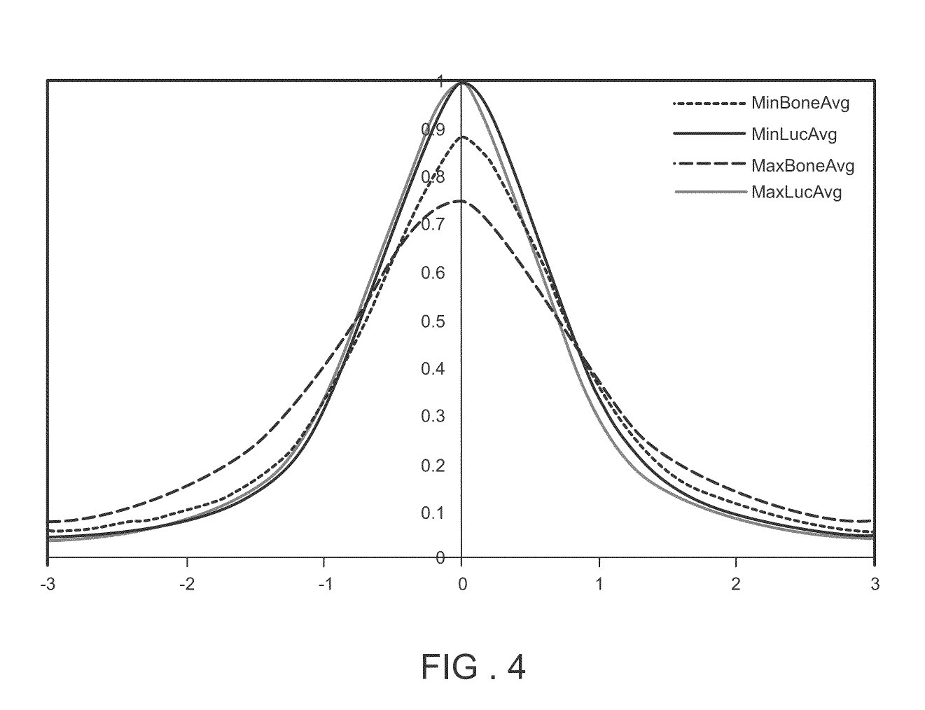

Referring to FIG. 4, a graph 400 of an example maximum angular width and minimum angular width of DEI reflectivity profiles using a bovine cortical bone sample, according to embodiments of the subject matter described herein is shown. Additionally, corresponding profiles taken through Lucite are also shown. The peak reflectivity through Lucite is normalized to one (1) and the reflectivity through the bone is normalized to the Lucite reflectivity.

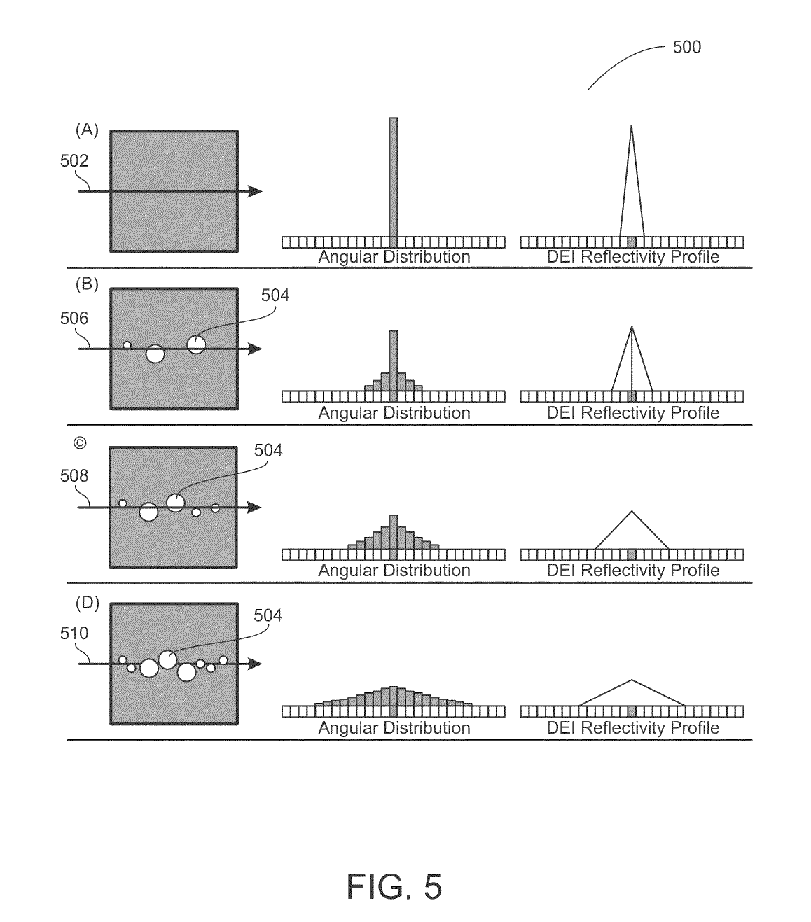

Referring to FIG. 5, a diagram 500 illustrates an X-ray beam passing through multiple refracting structures and the effect of multiple refractions on the measured reflectivity profile width, according to embodiments of the subject matter described herein. As an example, as an object with many structures is placed in the imaging field, the X-ray beam may be angularly diffused by the object (or structures within the object). FIG. 5 diagrams angular diffusion of the X-ray beam along with the corresponding reflectivity profile widths for when there are no refracting features in the X-ray beam 502 and with increasing numbers of refracting structures 504 along the beam path 506, 508, 510.

The amount of angular diffusion of the beam and angular shape of the beam diffusion are a function of the difference in index of refraction between the refracting structures and the bulk matrix in which the structures reside, the energy of the X-ray beam, and the shape and angular orientation of the structures with respect to the X-ray beam. The shape and the angular orientation of the structures and microstructures can be indicative of the strength, porosity, and fracture resistance of the object.

As an example, in young, healthy individuals, the trabeculae in their vertebrae are largely isotropic. In aged, osteoporotic individuals, the trabeculae transition from largely isotropic to largely anisotropic, with the trabeculae aligning with the axis of the spine. Thus, for someone at risk of developing osteoporosis, the degree of anisotropy within the vertebrae can be monitored with this new approach and a fracture risk stratification measure can be implemented with this approach.

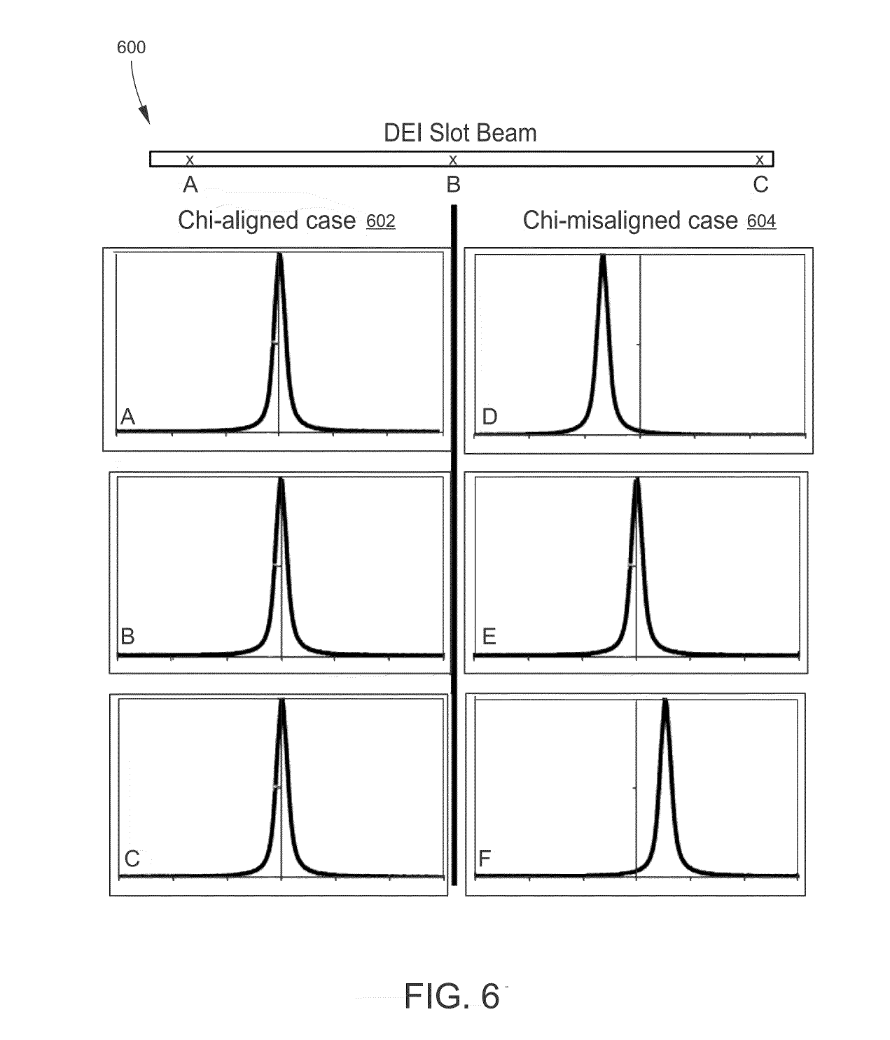

Referring now to FIG. 6, a diagram illustrating exemplary reflectivity profiles 600 of a chi-aligned 602 and chi-misaligned 604 X-ray beams are shown, according to embodiments of the subject matter described herein. FIG. 6 illustrates the reflectivity profiles, as a function of the angle theta, θ, for the case in which the chi-angle is fully aligned (chi-aligned 602 A-C), and for the case where the chi-angle, χ, is tilted away from the aligned chi-angle-position (chi-misaligned 604 D-F). It may be desired to initially align the χ-angle of the analyzer crystal and to not deviate from the χ-aligned angle. For the χ-aligned case 602, the peak position of the θ-reflectivity profile is substantially the same at all points in the DEI slot x-ray beam. For the χ-misaligned case 604, the peak position of the θ-reflectivity profile shifts as a function of the angle theta, θ.

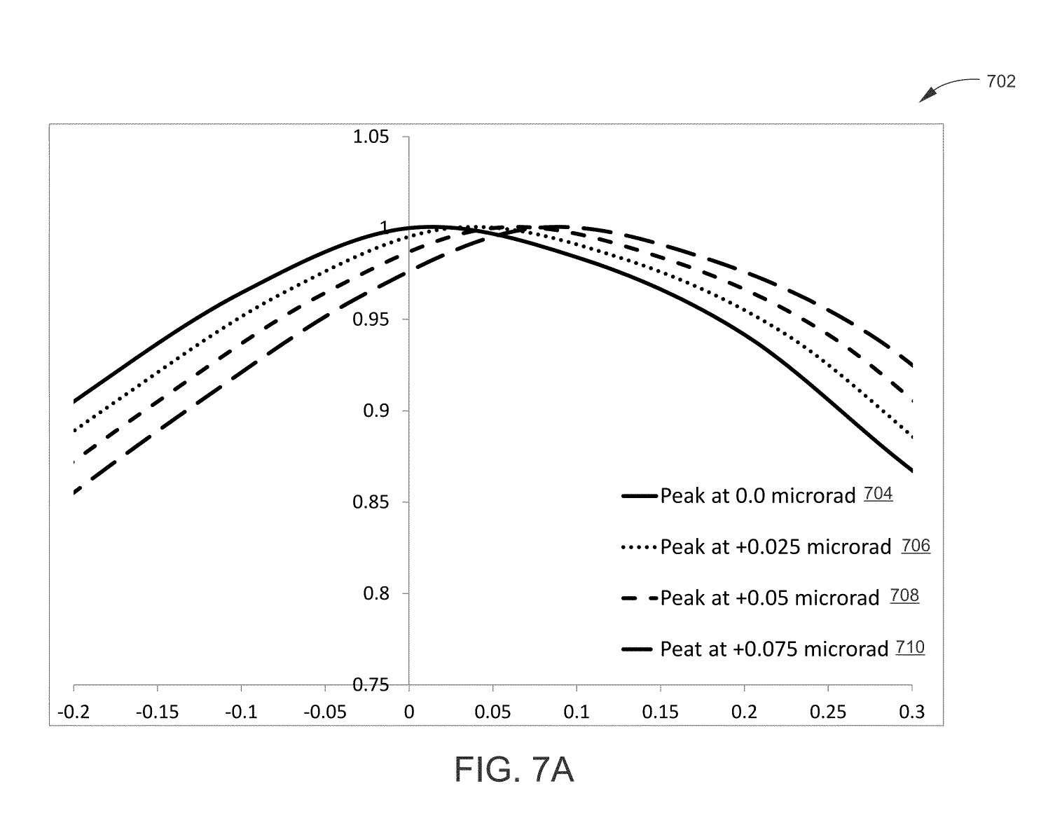

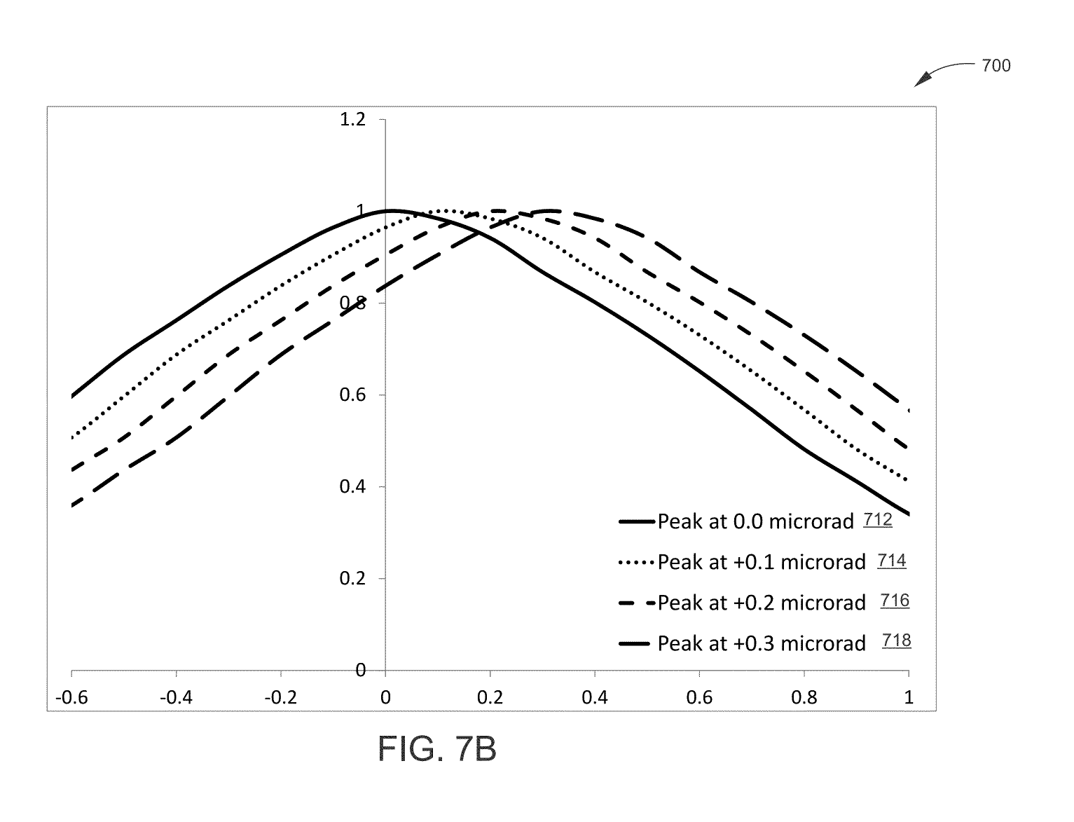

Referring to FIGS. 7A and 7B, is an illustration showing the effects of purposefully misaligning the χ-angle of the analyzer crystal by a predetermined amount, according to embodiments of the subject matter described herein. Purposefully misaligning the χ-angle may have two effects, explained in the two cases that follow, over an aligned χ. In Case A1 700, misaligning χ by a predetermined amount may allow an improvement to the χ-angle resolution of the DEI imaging system (high angular resolution case). In Case A2 702, fewer theta-angle positions for the analyzer crystal could be used to obtain a substantially complete reflectivity profile, which may reduce the reflectivity profile acquisition time (high speed reflectivity profile acquisition case). In the χ-aligned case the multiple-refraction signal may or may not significantly differ between neighboring pixels (the multiple-refraction signal is a slowly varying signal over the length-scale of the pixel width).

To implement either of the above cases, it is possible to first align the analyzer and monochromator crystals to be parallel, and then slightly misalign the analyzer crystal's χ-angle by a predetermined amount. The amount of χ-misalignment can be determined by a combination of the size of the pixels and the size-scale over which the multiple refraction signal remains constant. After misaligning χ, and before putting an object in the X-ray beam, a high angular resolution reflectivity profile, R(θ) may be obtained. For each point in the slot x-ray beam, the reflectivity profile may be fit to a curve (for example, a Gaussian), and the peak reflectivity position may be determined. This peak reflectivity as a function of position in the slot beam may serve as a lookup map for the subsequent reflectivity profiles. This approach may yield an accurate measure for the peak θ-position of the reflectivity profile.

As an example of the Case A1 700, if a feature of interest is at least 4 pixels wide, χ may be misaligned such that the peak θ-position of the analyzer crystal corresponds to N microradians for pixel one 704, N+0.025 microradians for pixel two 706, N+0.05 microradians for pixel three 708, and N+0.075 microradians for pixel four 710. If images are then obtained for every 0.1 microradians in θ, then the values from each pixel can be used to improve the theta angular resolution to 0.025 microradians. Thus, in this example, Case A1 700 provides a factor of 4× improvement in the angular resolution of the DEI system and reduces the error in measuring the width of the reflectivity profile.

As another example, Case A2 702 may have a feature of interest that is at least 4 pixels wide, χ may then be misaligned such that the peak θ-position of the analyzer crystal corresponds to N microradians for pixel one 712, N+0.1 microradians for pixel two 714, N+0.2 microradians for pixel three 716, and N+0.3 microradians for pixel four 718. If images are then obtained for every 0.4 microradians in theta, then the full reflectivity profile can be measured using four times fewer analyzer crystal θ-positions than a comparable angular resolution reflectivity profile obtained with an aligned chi-angle. This can reduce the acquisition time for a reflectivity profile by a factor of 4.

Case A1 700 and A2 702 may also be combined, for example, the feature of interest is 4 pixels wide, the χ could be misaligned such that the peak θ-position of the analyzer crystal corresponds to N microradians for pixel one, N+0.05 microradians for pixel two, N+0.1 microradians for pixel three, and N+0.15 microradians for pixel four. If images are then obtained for every 0.2 microradians in theta, then the full reflectivity profile can be measured with twice the initial (no χ-detuning) angular resolution while still reducing the acquisition time by a factor of 2.

With continued reference to FIG. 7, the reflectivity profile has utility because the width of the reflectivity profile is proportional to the number of refracting microstructures along the x-ray beampath, but the prior approach to measuring the reflectivity profile either required too long for image acquisition or was not sufficiently sensitive to small changes in the width of the reflectivity profile. The approach described herein can solve either of those problems (or both in combination). Beyond clinical utility of this approach, this can be used for industrial inspection. Any scenario in which it's advantageous to know how much microstructure is present can benefit greatly.

According to another aspect, the subject matter described herein can include a method for simultaneous acquisition of DEI reflectivity profiles at multiple X-ray energies. For a typical reflectivity profile, the monochromator and analyzer crystal can be set to the correct angle to reflect the X-rays that have the energy of the Kα1 and Kα2 emission lines of the x-ray tube source. While typically only the energies of the Kα1 and Kα2 emission lines are considered, the X-ray optics also can diffract x-rays with energies that correspond to the harmonic diffraction peaks. For example, if the silicon [333] plane of the monochromator/analyzer crystals are angled to diffract the Kα1/2 emission lines of tungsten, then the diffracted beam can also include X-ray with energies corresponding to the [111], [444], [555], etc. diffraction peaks for same angle as the 59.312 keV [333] diffraction peak. Thus, the diffracted x-ray beam can include bremsstrahlung photons of 59.312/3=19.77 keV (corresponding to the [111] diffraction peak), 59.312*4/3=79.083 keV (corresponding to the diffraction peak), and 59.312*5/3=98.853 (corresponding to the [555] diffraction peak). Because the brightness of the X-ray beam at the bremsstrahlung-only X-ray energies is already much lower than the brightness of the X-ray beam at the Kα1 and Kα2 energies, the lower energy (lower order harmonic), in this case just the 19.77 keV, [111] diffraction peak, may be attenuated out of the transmitted beam. The higher-order harmonics have higher energy than the Kα1 and Kα2 beams, and therefore will more readily transmit through the object in the imaging field. If an energy-resolving detector is used to obtain the DEI images, then reflectivity profiles corresponding to each of the [333], and [555] diffraction peaks can simultaneously be measured.

The width of the reflectivity profile is related to the number of refracting structures along the beam path, but the area under the reflectivity profile curve is related to total attenuation of the x-ray beam as it passes through object. If the attenuation of an X-ray beam is measured for two or more x-ray energies, then the object attenuation may be decomposed into a superposition of two attenuating materials (i.e. soft tissue and calcium). Through this decomposition of material, we can determine the amount of each of the attenuating material along the beam path. This is the same principal that is employed in dual-energy X-ray absorptiometry (DXA) for the measurement of bone mineral density.

This system is unique from existing D×A systems because the analyzer crystal absorbs the vast majority of scattered x-rays, which will increase the accuracy of the measurements for each energy. The major advancement over existing technology is that these systems and methods can allow for the simultaneous measurements of DXA and of microstructure along the beam path, thereby providing information both about the chemical composition of the object and information about the structural and microstructural properties of the object.

The reflectivity profile may have clinical utility because the width of the reflectivity profile is proportional to the number of refracting microstructures along the X-ray beam path. This may prove to be a diagnostically important measure for lungs and bone. In lungs, the collapse of alveoli in an injured lung region can lead to a reduced reflectivity profile width. In bone, degradation of microstructure corresponds to fracture risk, so a measure of bone microstructure will improve clinicians' ability to assess fracture risk. Simultaneous DXA and reflectivity profile widths can be important because it can provide information both about the chemical composition of the object and information about the structural and microstructural properties of the object. Beyond clinical utility of this approach, this can be used for industrial inspection or other scenarios in which it may be advantageous to know how much microstructure is present can benefit greatly.

According to another aspect, the subject matter described herein can include systems and methods for measuring the orientation direction and the degree of anisotropy in an object with multiple refracting structures. When an object with many structures is placed in the imaging field, the x-ray beam may be angularly diffused by the x-ray beam. The amount of angular diffusion of the beam and angular shape of the beam diffusion are a function of the difference in index of refraction between the refracting structures and the bulk matrix in which the structures reside, the energy of the X-ray beam, and the shape and angular orientation of the structures with respect to the X-ray beam.

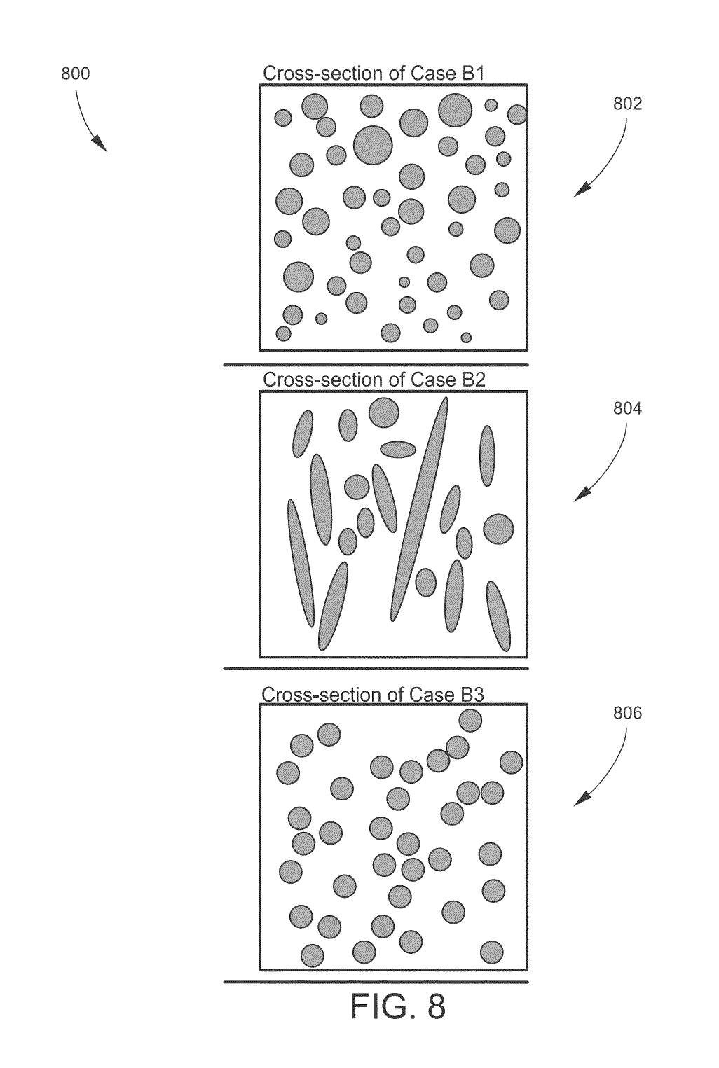

Referring to FIG. 8, an illustration 800 is provided of the exemplary effect shape and angular orientation of structures have on the X-ray beam, according to embodiments of the subject matter described herein. As an example, several types of structures along the beam path may be considered: Case B1 802 an array of spheres, Case B2 804 an array of cylinders with each cylinder's axis along an arbitrary direction, and Case B3 806 an array of cylinders with their axes aligned along the same direction.

In Case B1 802, if an X-ray beam passes through an array of spheres that differ in index of refraction from the bulk medium, then the X-ray beam will be diffused about its straight-line path. For randomly placed spheres along the beam path, the diffusion can have a Gaussian distribution, centered on the straight-line path and symmetric in angle about the straight-line path.

In Case B2 804, if an X-ray beam passes through an array of cylinders that are each randomly aligned (fully isotropic) and differ in index of refraction from the bulk medium, then the X-ray beam will be diffused about its straight-line path, the diffusion may have a Gaussian distribution, centered on the straight-line path and symmetric in angle about the straight-line path.

In Case B3 806, if an X-ray beam passes through an array of cylinders that are aligned parallel to one another (fully anisotropic) and differ in index of refraction from the bulk medium, then the x-ray beam will be diffused about its straight-line path, but that angular diffusion may only be non-zero in the plane perpendicular to the axis of the cylinders.

In present disclosure, systems and methods are provided for measuring the anisotropy and preferred angular orientation of structures through selective acquisition of DEI data.

Returning to Case B3 806 above, if the object was placed in the DEI X-ray beam such that the aligned axis of the cylinders was along an arbitrary direction in the plane perpendicular to the propagation direction of the center of the X-ray beam, then the measured reflectivity profile of the transmitted DEI beam, Rₜ(θ), can have a width, W, between a minimum of the intrinsic reflectivity profile width (the angular diffusion is fully misaligned to DEI's direction of sensitivity) and a maximum the convolution of the intrinsic reflectivity profile width with the angular diffusion map in Case B3 806 (the angular diffusion direction is aligned to DEI's sensitivity direction).

The next step is to rotate either the object or the DEI system about the center line of the beam propagation direction by an angle, a, and then to measure the reflectivity profile width again. The change in angle can be achieved either by rotating the DEI optical system or by rotating the object in the imaging field. Again, the measured reflectivity profile of the DEI X-ray beam may be between a minimum of the intrinsic reflectivity profile width (the angular diffusion is fully misaligned to DEI's direction of sensitivity) and a maximum the convolution of the intrinsic reflectivity profile width with the angular diffusion map in Case B3 806 (FIG. 8; the angular diffusion direction is aligned to DEI's sensitivity direction). This can be repeated for an array of angles, a, such that for each point in DEI images, there will be reflectivity profiles that are a function of θ and α, R(θ, α).

With continued reference to FIG. 8, the width of the reflectivity profile can then be measured for each angle, W(α). The angle, αₘₐₓ, that corresponds to the maximum width of the reflectivity profile, Wₘₐₓ(α), is 90 degrees away from the axis-direction for the cylinders. Likewise, the angle, αₘᵢₙ, that corresponds to the minimum width of the reflectivity profile, Wₘᵢₙ(α), is parallel to the axis-direction for the cylinders, and in this specific case, Wₘᵢₙ may be equal to Wᵢₙₜ, the intrinsic width of the reflectivity profile.

If W(α) were to be measured as above for Case B1 802 and B2 804, it would be noted that W is independent of α. For fully isotropic structures, W will not vary as a function of α. Many naturally occurring objects have cylinder-like structures or microstructures that reside on the continuum between Cases B2 804 (angular orientation of the structures is fully isotropic) and B3 806 (angular orientation of the structures is fully anisotropic). The degree of anisotropy (DA) can be calculated as:

$$DA = 1 - \frac{{W}_{min} - {W}_{int}}{{W}_{max} - {W}_{int}} \qquad Equation 3$$

DA has a value of zero, when Wₘᵢₙ=Wₘₐₓ, to a maximum of one, when Wₘₐₓ>>Wₘᵢₙ.

For example, there are rod-like trabeculae in trabecular bone, the rods preferentially align along the direction of load on the bone at that location, but the rods are not fully aligned. In this example, the rod-like trabeculae are slightly anisotropic (more similar to Case B2 804 than Case B3 806), thus W may vary as a function of α, but the maximum reflectivity profile width will be greater than, but not much, much greater than, the minimum measured reflectivity profile width (Wₘₐₓ is greater than Wₘᵢₙ, but Wₘₐₓ is not much, much greater than Wₘᵢₙ). In this case, the DA will approach zero as Wₘₐₓ approaches Wₘᵢₙ.

In this example, in regions where the load on the trabecular bone is uniform, then there is no preferred orientation direction for the trabeculae, and the maximum and minimum widths may be approximately equal, and both may be greater than the intrinsic width (Wₘₐₓ≅Wₘᵢₙ>Wᵢₙₜ). In this case, the DA may be close to zero.

While the above example cases only consider a single width measurement for each angle, α, reflectivity profile width images can be measured for each angle, α. Once the individual images are registered to one another (co-registered), the degree of anisotropy and the orientation direction (the value of α where W(α) is minimized) can be measured on a pixel-by-pixel basis in the image. Thus, with this novel method, the degree of anisotropy and the preferred angular orientation direction can be mapped within an object.

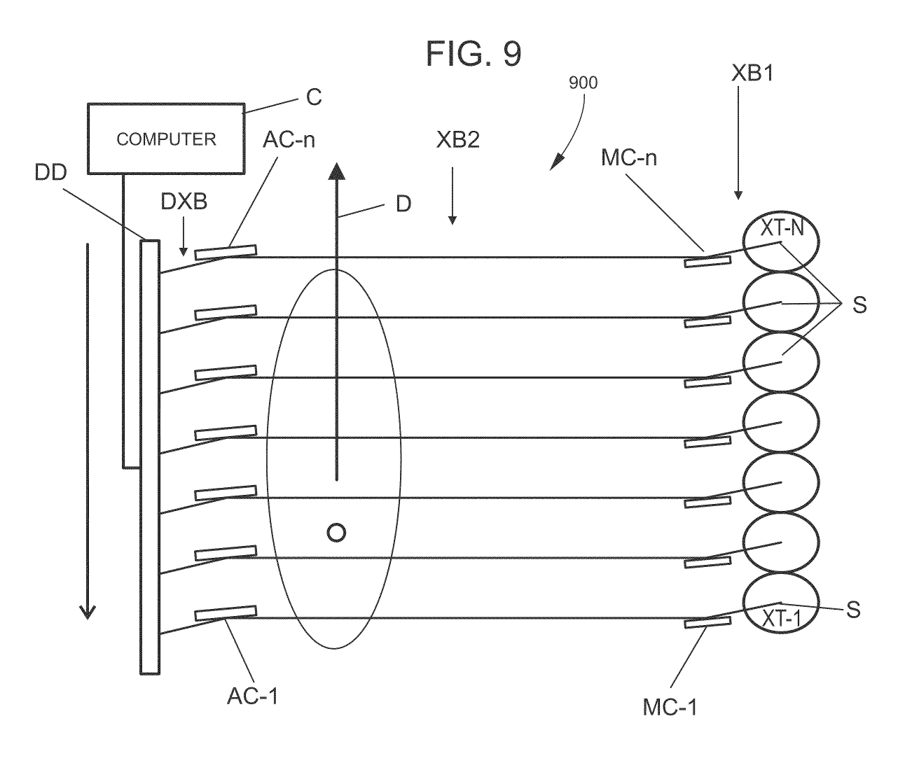

Referring now to FIGS. 9-19, a DEI system according to embodiments of the subject matter described herein can include multiple monochromator crystals for rejecting particular X-rays emitted by multiple X-ray small area sources. FIGS. 9-19, are schematic diagrams of different example DEI systems including multiple monochromator crystals and multiple small area sources according to embodiments of the subject matter described herein. The DEI systems are operable to produce images of an object by use of the X-ray beams generated by the multiple small area sources. The DEI systems can include multiple small area sources operable to produce a polychromatic X-ray beam, generally designated XB1. X-ray beams XB1 can include photons having different energies. In one example, the X-ray beams are generated by one or more tungsten X-ray tubes each having a small area source from which an X-ray beam. In another example, a system may include multiple X-ray tube sources that each provide one or more small area sources and may be used together for generating multiple X-ray beams.

Referring again to FIG. 9, a DEI system, generally designated 900, includes a number N X-ray tubes XT-1-XT-N, each including at least one small area source S, for generating multiple X-ray beams XB1. An array of collimators (not shown) may be positioned adjacent each small area source S for blocking a portion of each of X-ray beams XB1 that fall outside an angular acceptance window of respective monochromator crystals MC-1-MC-n. System 900 can also include other collimators positioned between small area sources XT-1-XT-N and monochromator crystals MC-1-MC-n for blocking a portion of X-ray beams XB1 that falls outside an angular acceptance window of the monochromator crystals MC-1-MC-n. The collimators can define a slit or hole through which a portion of X-ray beams XB1 can pass to monochromator crystals MC-1-MC-n. Further, the collimators can be made of any suitable material for blocking X-ray beams such as lead.

The monochromator crystals MC-1-MC-n can be configured to select a predetermined energy of a portion of X-ray beams XB1 incident thereon. In one example, a monochromator crystal is a silicon [333] monochromator crystal adapted to reject the majority of photons of its respective X-ray beams that do not have a desired energy. For the case of a tungsten X-ray tube, there can be a range of beam energies that are reflected by the silicon monochromator crystal. In this case, the characteristic emission lines of the X-ray beams are 59.13 keV (Kα1) and 57.983 (Kα2), and the Bremsstrahlung radiation that falls within the narrow angular acceptance window of the monochromator crystal. The brightness of the bremsstrahlung radiation is several orders of magnitude less than the two Kα emission lines.

An X-ray beam may be scattered by its respective monochromator crystal in several different directions. Another array of collimators (not shown) may be positioned between the monochromator crystals MC-1-MC-n and the object O for blocking a portion of the X-ray beam that falls outside an angular acceptance window of its corresponding analyzer crystal, one of analyzer crystals AC-1-AC-n. Each collimator can define a slit or hole through which a portion of one of the X-ray beams can pass towards its analyzer crystal for interception by the analyzer crystal.

The analyzer crystals AC-1-AC-n can be rotated for measuring the amount of radiation traveling in a particular direction. The angular reflectivity function of the crystal system is called the intrinsic rocking curve, and this property is used to generate image refraction contrast. If an X-ray photon is deviated towards the peak of the rocking curve, its reflectivity, and thus intensity will increase. If an object feature causes a photon to be deflected down the rocking curve, or away from the peak reflectivity position, it will cause a reduction in intensity.

A sample or object O can be imaged in air or immersed in a coupling medium, such as water. The use of a coupling medium can be used to reduce the index gradient between the air and the object O to be imaged, thus allowing the incident X-rays to pass into the object without experiencing significant refraction at the air-object interface. This is not necessary for most objects, but it is an application of the DEI method and can be used to improve the internal contrast of an object.

In one example, a monochromator crystal is a symmetric crystal which is narrow in one dimension. A symmetric crystal's lattice planes (the atomic layers that contribute to diffracting the X-ray beam) are parallel to the surface of the crystal. A symmetric crystal preserves the vertical height of the corresponding X-ray source in the incoming beam. In comparison, an asymmetric crystal modifies the divergence and size of the incoming beam. In this example of a monochromator crystal being a symmetric crystal, two-dimensional imaging of large imaging fields (e.g., imaging fields of about 25 cm by 20 cm) can be achieved by scanning a sample object and a detector using a symmetric crystal. One exemplary advantage of a symmetric crystal over an asymmetric crystal is that the asymmetric crystal requires a large monochromator crystal to prepare the imaging beam (e.g., selecting and collimating X-rays), imposing a severe limitation on the perfection of the large crystal. Further, the size of an asymmetric crystal increases with increasing X-ray beam energy, thus making it impractical for X-rays of about 59.13 keV. In contrast, for example, a symmetric monochromator crystal used in accordance with the subject matter described herein can utilize 59.13 keV X-rays with a modest sized crystal of about 30 mm in length. An advantage, over single-beam DEI, of the system and methods proposed disclosed herein, with multiple sources, is that this scan range can be greatly reduced, because of much better spatial coverage of the beams (i.e. if you have a required 25 cm scan range, and 10 beams, then the object will only have to be scanned through a range of 2.5 cm).

Referring again to FIG. 9, the object O can be positioned in the path of X-ray beams XB2 (the X-ray beams resulting for the interaction of X-ray beams XB1 with the monochromator crystals MC-1-MC-n) by, for example, a scanning stage (not shown) for imaging of the object O. The object O can be scanned in a direction D, which is approximately perpendicular to the direction of X-ray beams XB2. During scanning of the object O, X-ray beams XB2 can pass through object O and can be analyzed by analyzer crystals AC-1-AC-n, which can be silicon [333] crystals that match monochromator crystals MC-1-MC-n. X-ray beams XB2 incident on analyzer crystals AC-1-AC-n can each diffract (resulting in diffraction X-ray beams, generally designated DXB) for interception by a digital detector (or image plate) DD. Digital detector DD can detect the diffracted X-ray beams DXB and generate electrical signals representative of the intercepted X-ray beams DXB.

The electrical signals can be communicated to a computer C for image analysis and display to an operator. The computer C can be configured to generate an absorption image, an image showing refraction effects, and an image depicting ultra-small-angle scattering, the types of which are described in more detail below.

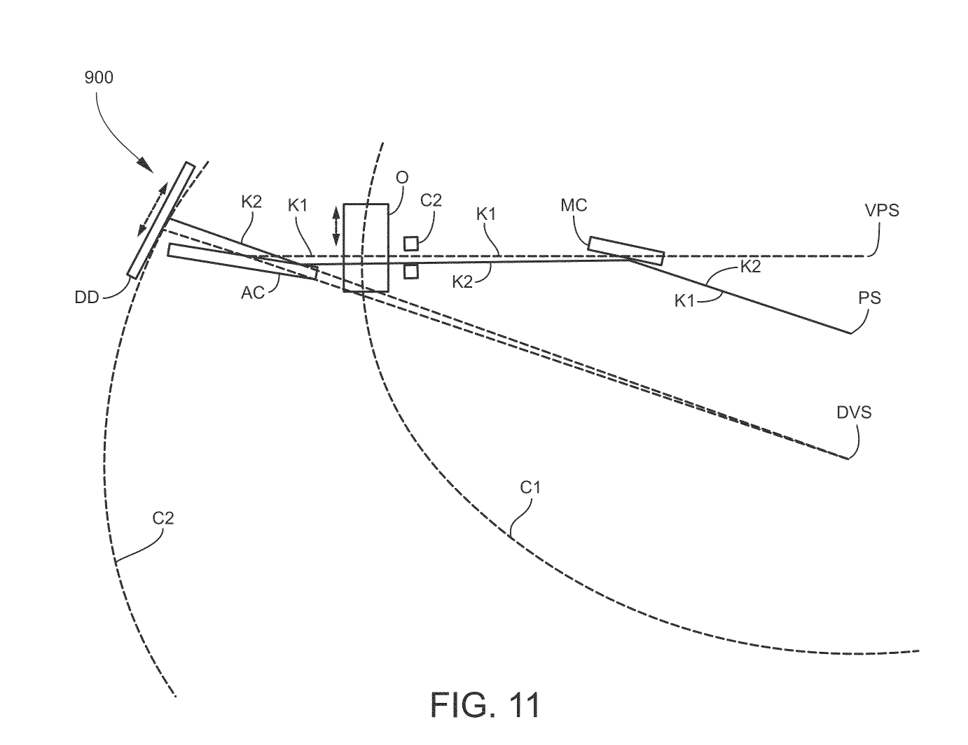

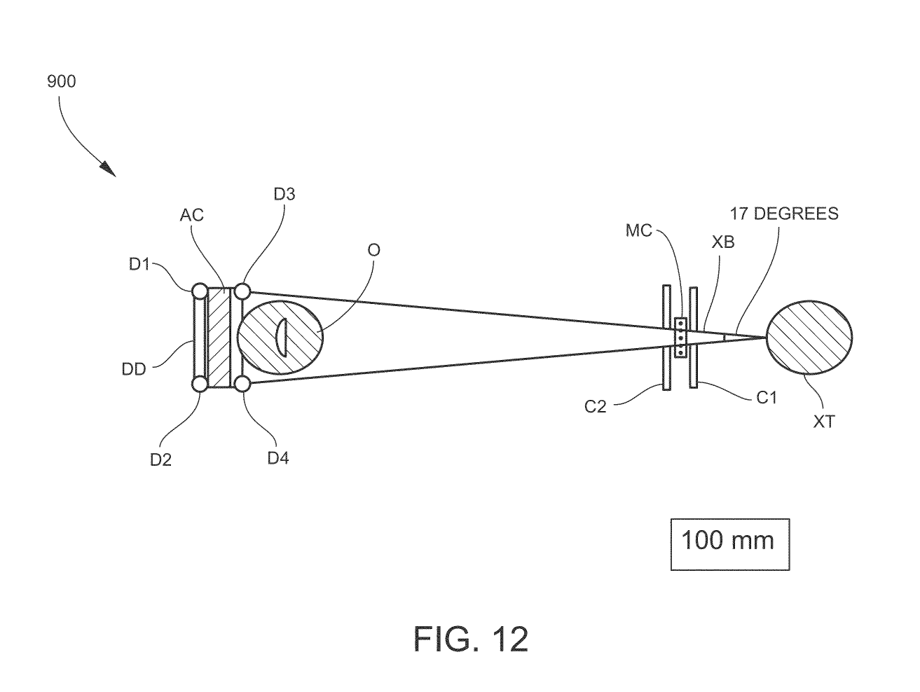

The monochromator crystals can propagate their respective x-ray beams as a horizontally-divergent (FIG. 12) and partially vertically divergent (see FIG. 11) fan beam. The fan beam can be collimated with one or more collimators to shield against undesired X-rays, resulting in clear DEI images and low subject dose. In contrast to a two-dimensional beam, a fan beam can be more readily controlled for the shielding of undesired X-rays.

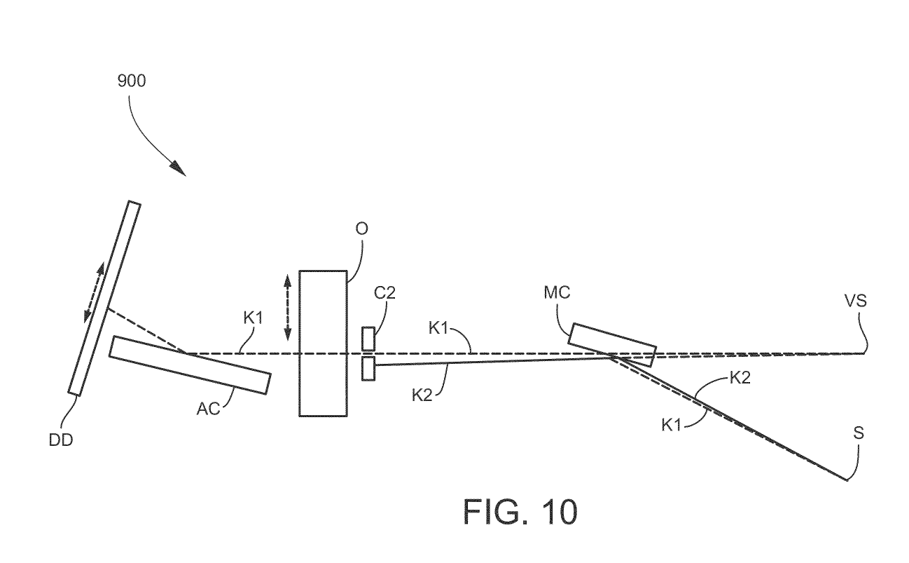

Referring now to FIGS. 10 and 11, the DEI system 900 is shown in different operation modes. For clarity, the X-ray beam generated by only one small area source S is shown. Characteristic emission lines Kα1 K1 and Kα2 K2 of the X-ray beam are generated by small area source S. Emission lines Kα1 K1 and Kα2 K2 originate from the same small area source S. As stated above, monochromator crystal MC rejects the majority of photons of the X-ray beam that do not have the desired energy. In this case, emission lines Kα1 K1 and Kα2 K2 and bremsstrahlung radiation pass monochromator crystal MC and are redirected towards an analyzer crystal AC as shown.

Collimator C2 is positioned in a path of emission lines Kα1 K1 and Kα2 K2. Collimator C2 defines an adjustable slit through which emission lines can be selectively passed towards analyzer crystal AC. In the first operational mode shown in FIG. 10, the slit is adjusted for an aperture of the vertical size of the X-ray source at a distance of about 400 mm from the small area source S, and positioned such that emission line Kα1 K1 passes collimator C2 and Kα2 K2 is blocked. Thus, collimator C2 removes all X-rays except for the X-rays from emission line Kα1 K1 and a very narrow range of bremsstrahlung radiation. In this mode, the beam is not vertically divergent and thus the object O and detector DD are scanned at the same scanning speed, in opposite directions. This mode yields a maximum possible out-of-plane resolution (the direction of DEI's contrast), but at the cost of removing a portion of the X-rays from the X-ray beam, thereby necessitating increased exposure time. The virtual small area source for the object O is designated VS.

Referring now to FIG. 11, in the second operational mode, emission lines Kα1 K1 and Kα2 K2 and the bremsstrahlung radiation at nearby energies are passed through the collimator C2. The slit of collimator C2 is adjusted for an aperture of about 2.0 mm at a distance of about 400 mm from the small area source S and positioned such that emission lines Kα1 K1 and Kα2 K2 and the bremsstrahlung radiation passes collimator C2. In this mode, the beam divergence is taken into account. In order to avoid image blurring, the object O and detector DD can be scanned at the same angular speed. The relative scanning speeds of detector DD and the sample stage on which the object O is placed can be determined by the source-to-object distance and the source-to-detector distance (where the distances are taken along the beam path). The beam divergence in this mode can lead to lower resolution out-of-plane, but this mode has the advantage of passing more X-rays and thus allows for a faster exposure time. The virtual small area source for detector DD is designated DVS. Circle portions C1 and C2 are centered at the virtual source points for the object O and detector DD, respectively.

Further, in one embodiment of using the second mode, the Bremsstrahlung radiation at x-ray energies that are different from the K alpha lines can be captured. Thus, in this embodiment, the system is tunable in x-ray energy and is not limited to the characteristic emission energies. This functionality can be achieved by changing the incident angle of the monochromator crystal and the analyzer crystal. In one example, this functionality can be achieved by changing the incident angle to 11.4 degrees, following the Bragg's law, and replacing the Copper filter with an Aluminum filter. In this example, imaging can occur at 30 keV x-ray energy. X-ray energies lower than the Tungsten emission line energies can be utilized for relatively thin objects.

In one example, the copper filter can be configured to remove about 19 keV bremsstrahlung radiation for reducing or eliminating unwanted crystal reflections and harmonics. Images have the potential to be degraded without this filtering.

FIG. 12 is a top schematic view illustrating the DEI system 900 of FIG. 9 according to an embodiment of the subject matter described herein. For clarity, the X-ray beam XB generated by only one small area source of an X-ray tube is shown. Referring to FIG. 12, X-ray beam XB are generated by a source of X-ray tube XT. Collimators C1 and C2 block the horizontal spread of the portion of X-ray beam XB to define the angular spread of the X-ray beam XB and its horizontal size at the object O position. The portion of X-ray beam XB that passes through collimators C1 and C2 is the X-ray beam portion that passes through slits in the collimators.

The DEI system 900 can include right and left post-analyzer crystal sodium iodide detectors D1 and D2, respectively, and right and left post-monochromator crystal sodium iodide detectors D3 and D4, respectively. Detectors D3 and D4 are used to ensure alignment of the monochromator crystals (MC) and detectors D1 and D2 are used to ensure analyzer crystal (AC) alignment. These detectors are used to measure the intensity of the diffracted X-ray beam being emitted from the monochromator crystal MC, or the analyzer AC. For system alignment, detectors D1 and D2 are placed in the post analyzer crystal AC X-ray beam XB. If the analyzer crystal is not tuned to the desired angle, the intensity measured by the detectors D1 and D2 will show this and the system can be adjusted. The same is true for the detectors in the post-monochromator crystal MC X-ray beam XB. In addition, detectors D1-D4 can be used to measure X-ray beam XB in real time and adjust the analyzer crystal, D1 and D2, chi (angle as measured about the axis along the X-ray beam path) or monochromator crystal chi, D3 and D4. The use of these detectors to set, measure, and adjust the analyzer crystal AC and monochromator crystal MC can be important for successful DEI image acquisition.

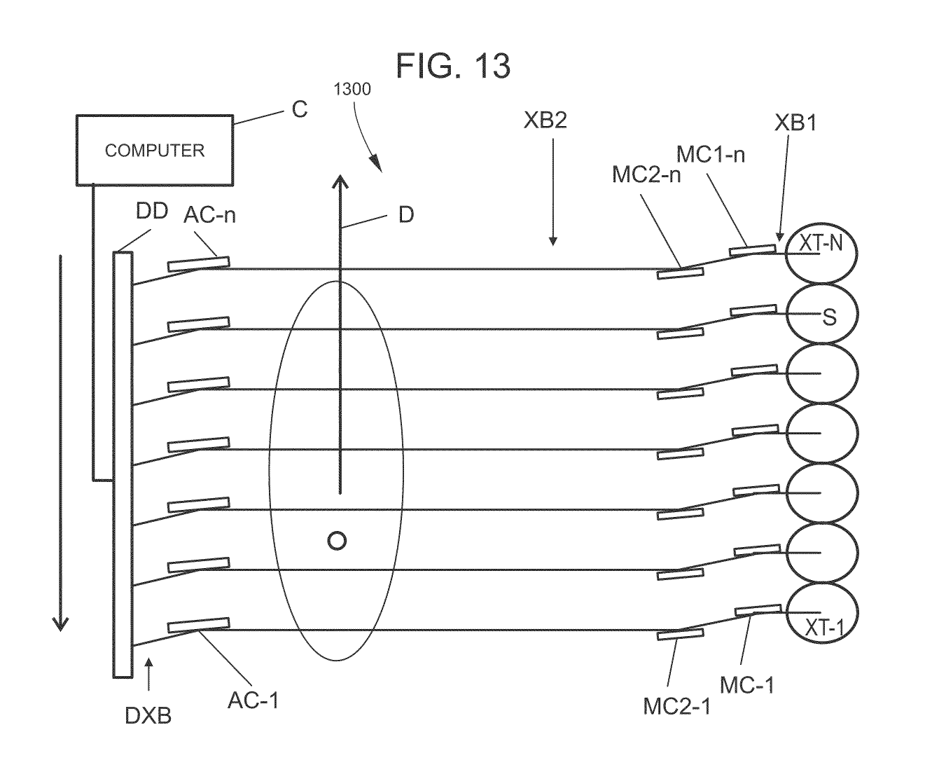

Referring now to FIG. 13, another example DEI system 1300 for detecting an image of the object O according to an embodiment of the subject matter disclosed herein is shown. The DEI system 1300 is similar to DEI system 100 shown in FIG. 9 except that DEI system 1300 includes a second set of monochromator crystals MC2-1-MC2-n positioned downstream from a first set of monochromator crystals MC1-1-MC1-n.

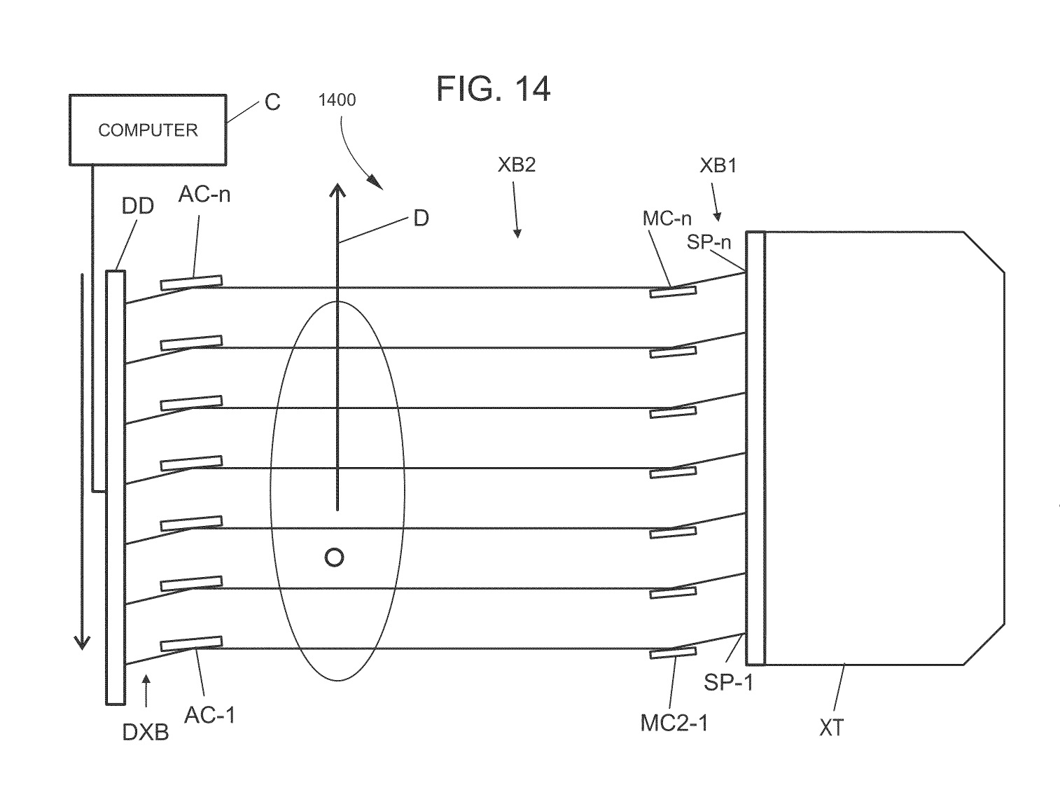

Referring now to FIG. 14, another example DEI system 1400 for detecting an image of the object O according to an embodiment of the subject matter disclosed herein is shown. DEI system 1400 is similar to DEI system 900 shown in FIG. 9 except that, rather than the use of multiple X-ray tubes XT-1-XT-N, system 1400 includes a single X-ray tube XT having multiple source points SP-1-SP-n, each capable of functioning as a small area source. Therefore, X-ray tube XT can produce a plurality of X-ray beams, generally designated XB1.

The DEI system 1400 shown in FIG. 14 also includes a collimator array CA, although the system may not include this component in another embodiment. Without the collimator array CA, the X-ray beams XB1 may be generated by small area sources at the X-ray tube XT. With the collimator array CA as shown in FIG. 14, a line beam, or even a large area X-ray beam produced by a large area X-ray beam source can be used in combination with the collimator array CA to generate a series of small area sources at the slits of the collimator array.

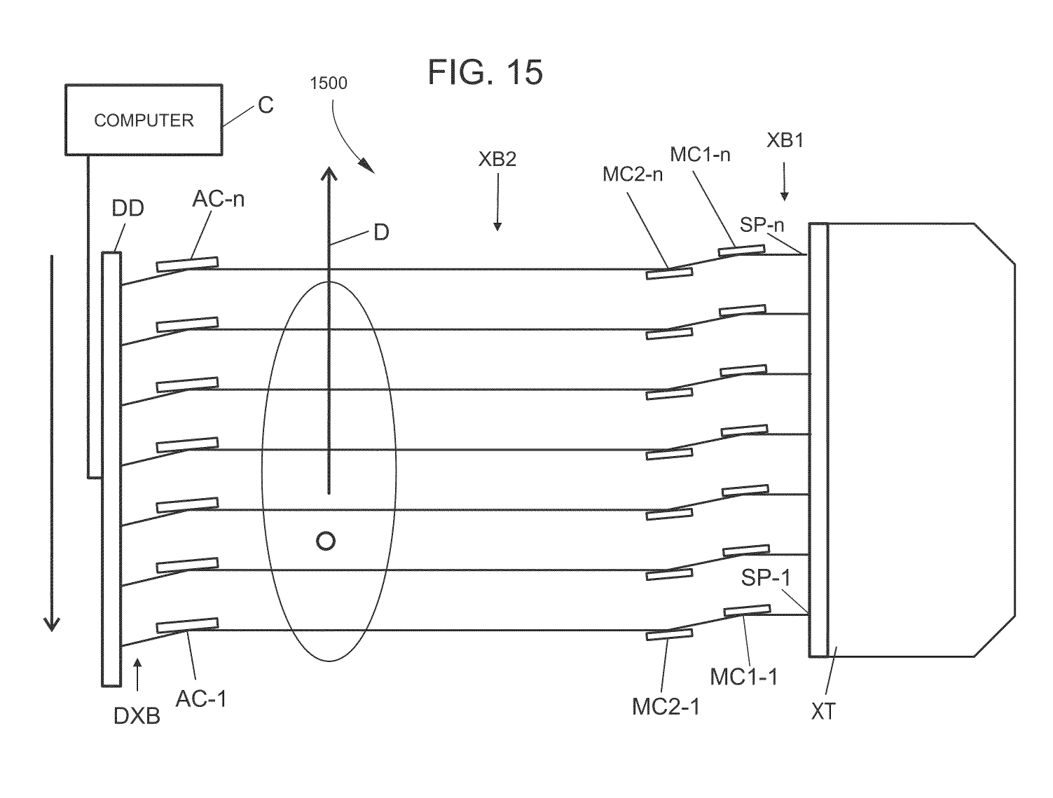

Referring now to FIG. 15, another example DEI system 1500 for detecting an image of the object O according to an embodiment of the subject matter disclosed herein is shown. DEI system 1500 is similar to DEI system 1300 shown in FIG. 13 and DEI system 1400 shown in FIG. 14. Similar to system 1300 shown in FIG. 13, system 1500 includes monochromator crystals MC1-1-MC1-n and MC2-1-MC2-n. Further, similar to system 1400 shown in FIG. 14, system 1500 includes a single X-ray tube XT having multiple source points SP-1-SP-n, each capable of functioning as a small area source for producing X-ray beams XB1. The system may include the collimator array CA or not as described with respect to FIG. 14.

According to one aspect, the subject matter described herein can include a method for detecting an image of an object by providing a plurality of small area sources. A plurality of first X-ray beams can be generated by using the small area sources. A plurality of monochromator crystals can be positioned to intercept the plurality of first X-ray beams such that a plurality of second X-ray beams each having predetermined energy levels is produced. Further, an object to be imaged can be positioned in paths of the second X-ray beams for transmission of the second X-ray beams through the object and emitting from the object a plurality of transmission X-ray beams. The X-ray beams may be directed at angles of incidence upon a plurality of analyzer crystals. Further, an image of the object can be detected based upon beams diffracted from the analyzer crystals. These systems and methods can be advantageous, for example, because they can provide extremely low dose in medical applications, fast scan times, high resolution, and relatively low operation and build costs. Further, for example, these systems can be constructed into a compact unit and be readily usable in clinical and industrial applications. Additional description about these systems and related methods are described in further detail herein.

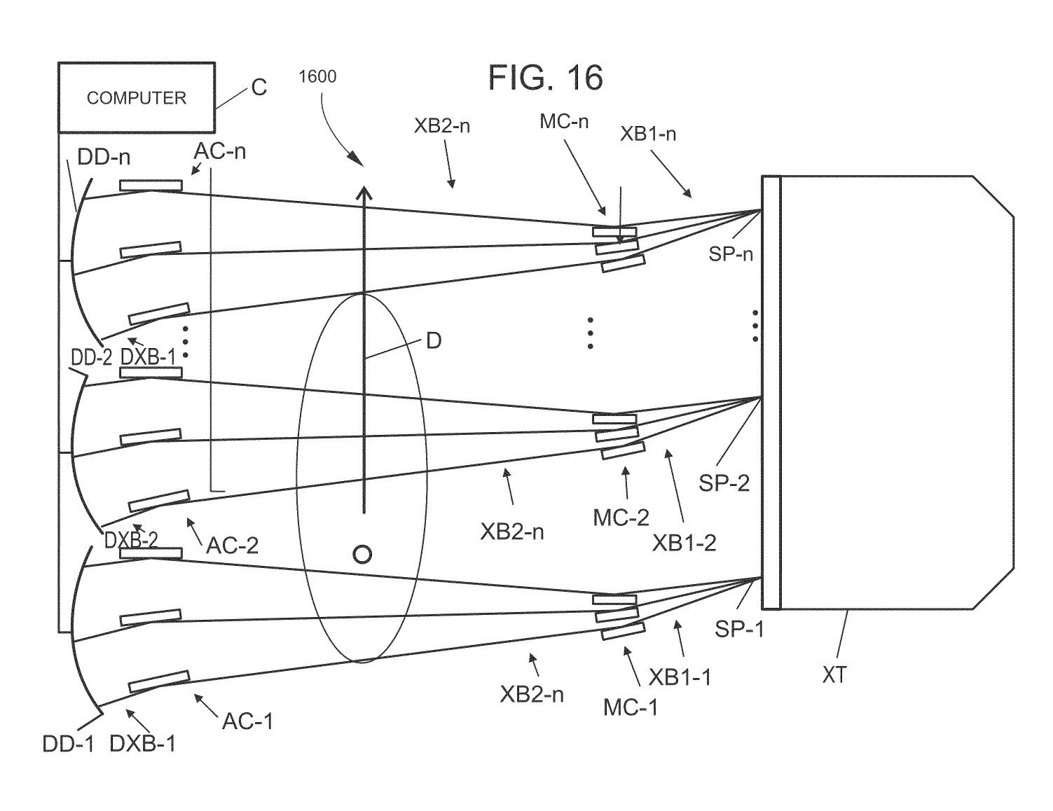

Referring now to FIG. 16, another example DEI system 1600 for detecting an image of the object O according to an embodiment of the subject matter disclosed herein is shown. DEI system 1600 is similar to DEI system 1400 shown in FIG. 146 except that the source points SP-1-SP-n of system 1600 each emit an X-ray beam XB that fans out toward sets of monochromator crystals MC-1-MC-n. For example, source points SP-1 and SP-n emit fanning X-ray beams, generally designated XB1-1 and XB1-n, respectively, directed to the sets of monochromator crystals MC-1 and MC-n, respectively. In turn, X-ray beam sets XB2-1-XB2-n, originating from the monochromator crystals, are directed towards the analyzer crystal sets AC-1-AC-n. The system may include the collimator array CA or not as described with respect to FIG. 14.

System 1600 includes a plurality of digital detectors DD-1-DD-n each configured to receive respective, diffracted X-ray beams DXB-1-DXB-n from the analyzer crystal sets AC-1-AC-n. Computer C is operable to receive electrical signals from the digital detectors DD-1-DD-n for generating an image of the object O.

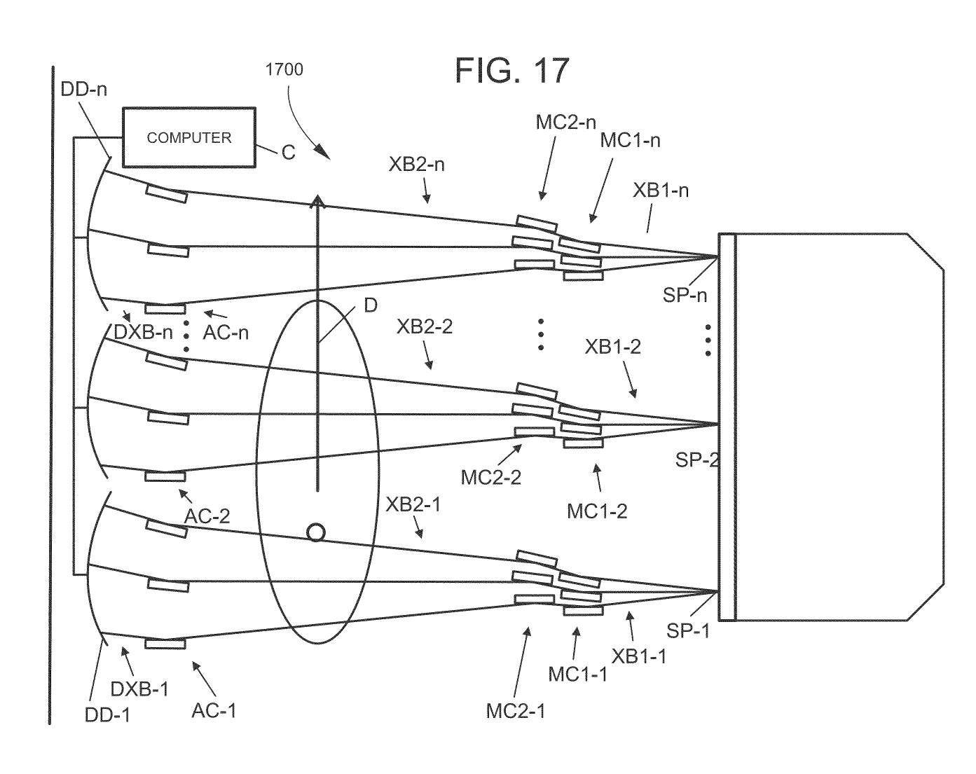

Referring now to FIG. 17, another example DEI system 1700 for detecting an image of the object O according to an embodiment of the subject matter disclosed herein is shown. DEI system 1700 is similar to DEI system 800 shown in FIG. 16 except that system 1700 includes monochromator crystals MC1-1-MC1-n and MC2-1-MC2-n similar to DEI system 1300 shown in FIG. 13. The system may include the collimator array CA or not as described with respect to FIG. 14.

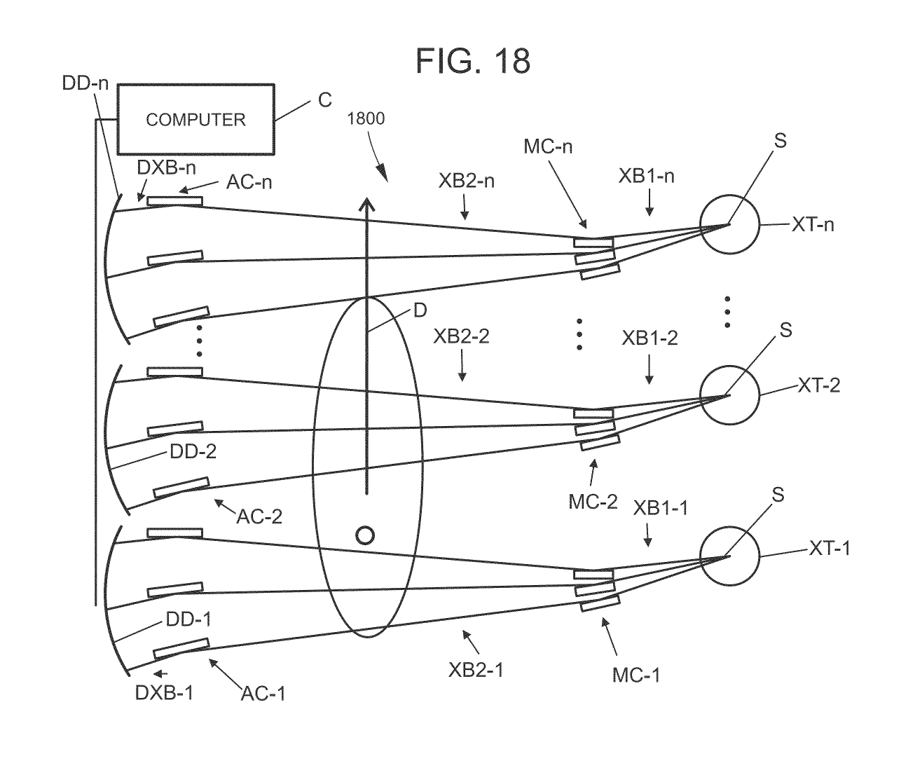

Referring now to FIG. 18, another example DEI system 1800 for detecting an image of the object O according to an embodiment of the subject matter disclosed herein is shown. DEI system 1800 is similar to DEI system 1600 shown in FIG. 16 except that system 1800 includes X-ray tubes XT-1-XT-n similar to the DEI system 1300 shown in FIG. 13.

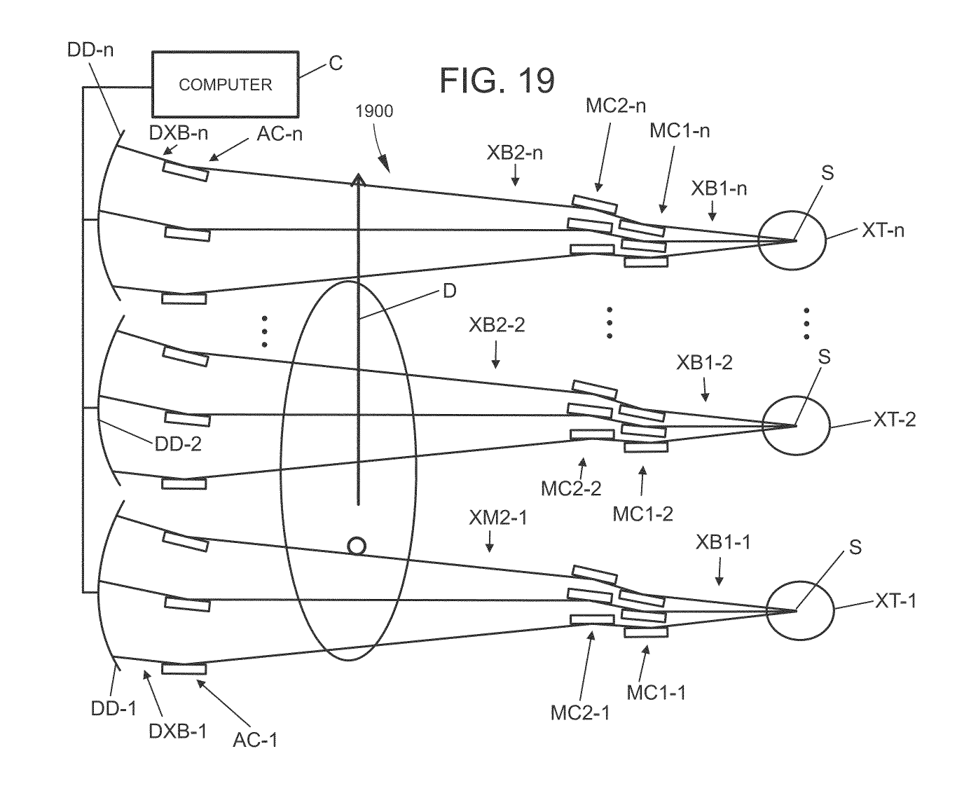

Referring now to FIG. 19, another example DEI system 1900 for detecting an image of the object O according to an embodiment of the subject matter disclosed herein is shown. DEI system 1900 is similar to DEI system 1700 shown in FIG. 17 except that the source points originate from different X-ray tubes XT-1-XT-n similar to the DEI system 1300 shown in FIG. 13.

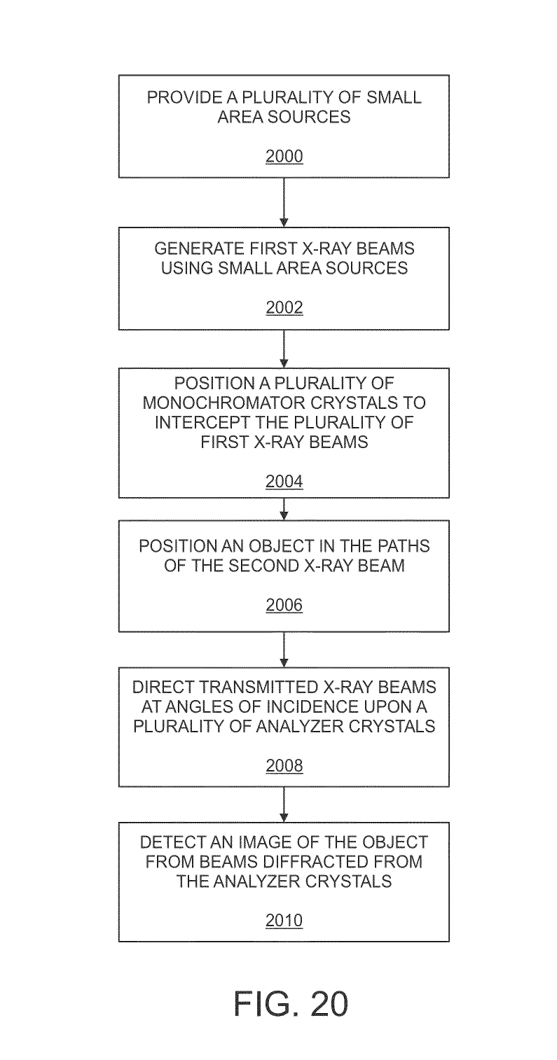

FIG. 20 is a flow chart illustrating an exemplary process for imaging object O by use of a DEI system, such as one of the DEI systems shown in FIGS. 9-19, according to an embodiment of the subject matter described herein. Referring to FIG. 20, in step 2000, a plurality of small area sources are provided. For example, the small area sources S of the X-ray tubes XT-1-XT-N shown in FIG. 1 may be provided in a DEI system.

In step 2002, a plurality of first X-ray beams may be generated using the small area sources. For example, the small area sources S of the X-ray tubes XT-1-XT-N shown in FIG. 9 may generate X-ray beams XB1.

A plurality of monochromator crystals, such as the monochromator crystals MC-1-MC-n shown in FIG. 9, may each be positioned to intercept a respective one of the first X-ray beams such that a plurality of second X-ray beams each having predetermined energy levels is produced (step 2004). For example, a surface of each of the monochromator crystals MC-1-MC-n shown in FIG. 9 can be positioned in the path of its respective X-ray beam for intercepting the beam. Each monochromator crystal can be adapted to reject the majority of photons of its respective X-ray beam that does not have a desired energy. Thus, a resulting second set of X-ray beams (e.g., X-ray beams XB2 shown in FIG. 9) can be produced that has a narrow range of X-ray energies. In one example, a surface of each monochromator crystal can be positioned at an angle of between about 5 degrees and 20 degrees with respect to a path of its respective X-ray beam incident upon the surface of the monochromator crystal. In this example, these angles may be used for [333] reflection. Alternatively, other suitable angles may be used in the positioning of the surface of monochromator crystal. In another example, a surface of each monochromator crystal can be positioned at an angle of between about 1 degree and 20 degrees with respect to a path of its respective X-ray beam incident upon the surface of monochromator crystal MC. If both [333] and [111] reflections are used, the angular range can be between about 1 degree and about 40 degrees for the energy range of 10 to 70 keV.

In step 2006, an object can be positioned in the paths of the second X-ray beams for transmission of the second X-ray beams through the object and emission from the object a plurality of transmission X-ray beams. For example, the object O shown in FIG. 9 can be positioned on a scanning stage for movement of the object O into the pathway of the second X-ray beams XB2.

In step 2008, the transmitted X-ray beam can be directed at angles of incidence upon analyzer crystals. For example, analyzer crystals AC-1-AC-n shown in FIG. 9 can be positioned in the paths of the transmitted X-ray beams and at an angle for intercepting the transmitted X-ray beams at angles of incidence. At least a portion of each beam intercepting a respective one of analyzer crystals AC-1-AC-n can be diffracted towards a detector, such as detector DD.

In step 2010, an image of object O can be detected from the beams diffracted from the analyzer crystal AC-1-AC-n. For example, detector DD can detect the diffracted beam from the analyzer crystals. The diffracted beams can be detected by one of the following exemplary detectors: a detector configured to digitize a detected image; a radiograph film; and an image plate. In one example, the image of an object can be detected from beam diffracted from analyzer crystals at a peak of a rocking curve of the analyzer crystals and/or near a peak of a rocking curve of the analyzer crystals. The detected image can be processed and presented to a user via a display of a computer.

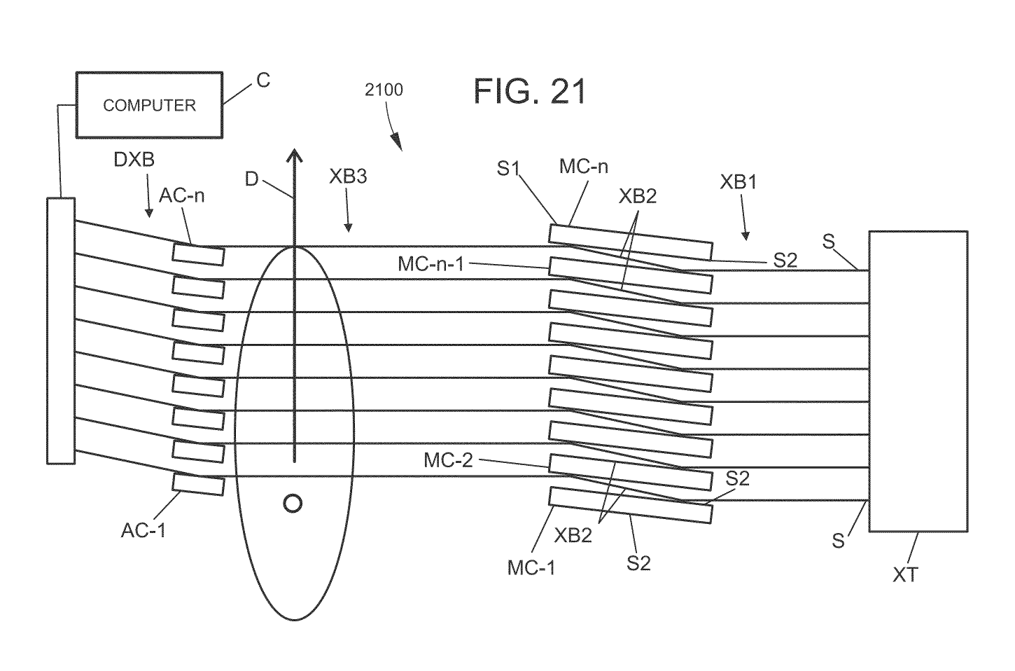

FIG. 21 is a schematic diagram of another example DEI system 2100 that can utilize facing sides of adjacent monochromator crystals for detecting an image of an object according to an embodiment of the subject matter described herein. The DEI system 2100 is similar to DEI system 1400 shown in FIG. 14 except that the DEI system 2100 utilizes facing sides of adjacent monochromator crystals MC-1-MC-n for detecting an image of an object. For example, an X-ray generation device, such as X-ray tube XT, can generate multiple X-ray beams XB1 that are intercepted by sides S1 of monochromator crystals MC-1-MC-n-1 for producing a plurality of X-ray beams XB2. The X-ray beams XB2 are directed to sides S2 of monochromator crystals MC-2-MC-n for producing X-ray beams XB3, which can be substantially parallel to X-ray beams XB1. An object can pass through X-ray beams XB3, and the transmitted X-ray beams intercepted by analyzer crystals for downstream processing as described in further detail herein. The system may include the collimator array CA or not as described with respect to FIG. 14.

It is noted that a DEI system, such as the system shown in FIG. 21, can have more than two reflections on the monochromator crystals per X-ray beam. For example, an X-ray beam can be directed from a source to a side of a monochromator crystal for a first reflection towards a facing side of another monochromator crystal. The X-ray beam can then be reflected between the sides of the monochromator crystals for any number of times before the X-ray beam finally exits the monochromator crystals towards downstream DEI system components.

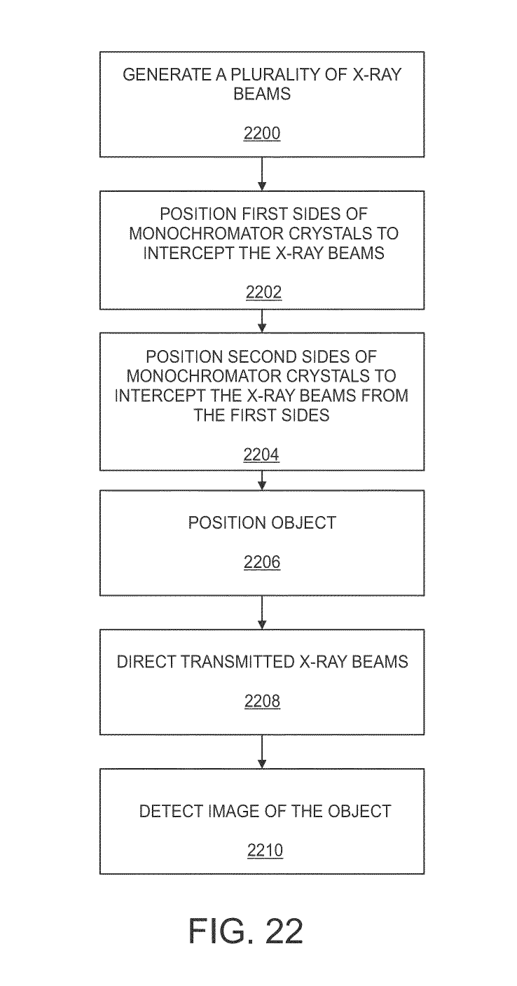

FIG. 22 is a flow chart illustrating an exemplary process for imaging an object by use of a DEI system, such as the DEI system 2100 shown in FIG. 21, according to an embodiment of the subject matter described herein. Referring to FIG. 22, in step 2200, a plurality of first X-ray beams may be generated by X-ray tube XT. Sides S1 of monochromator crystals MC-1-MC-n-1 are positioned to intercept a respective one of the first X-ray beams such that a plurality of second X-ray beams each having predetermined energy levels is produced (step 2202). For example, a surface S1 of each of the monochromator crystals can be positioned in the path of its respective X-ray beam for intercepting the beam. Each monochromator crystal can be adapted to reject the majority of photons of its respective X-ray beam that does not have a desired energy. Thus, a resulting second X-ray beam XB2 can be produced that has the predetermined energy level. In one example, a surface of each monochromator crystal can be positioned at an angle of between about 5 degrees and 20 degrees with respect to a path of its respective X-ray beam incident upon the surface of the monochromator crystal. In this example, these angles may be used for [333] reflection. Alternatively, other suitable angles may be used in the positioning of the surface of monochromator crystal. In another example, a surface of the monochromator crystal can be positioned at an angle of between about 1 degrees and 20 degrees with respect to a path of its respective X-ray beam incident upon the surface of the monochromator crystal. In another example, a surface of each monochromator crystal can be positioned at an angle of between about 1 degree and 20 degrees with respect to a path of its respective X-ray beam incident upon the surface of monochromator crystal MC. If both [333] and [111] reflections are used, the angular range can be between about 1 degree and about 40 degrees for the energy range of 10 to 70 keV.

In step 2204, the second sides S2 of the monochromator crystals MC1-MC-n are positioned to intercept the second X-ray beams XB2 for producing the third X-ray beams, generally designated XB3. An object O can be positioned in the paths of the third X-ray beams XB3 for transmission of the third X-ray beams XB3 through the object and emission from the object transmission X-ray beams (step 2206).

In step 2208, the transmitted X-ray beams can be directed at angles of incidence upon the analyzer crystals AC1-AC-n. Further, in step 2210, an image of the object can be detected from the diffracted X-ray beams DXB.

In another example of detecting the image of the object, a first angle image of object can be detected from first diffracted beams emitted from analyzer crystals positioned at a first angular position. The first angle image of the object can be detected at a low rocking curve angle setting of the analyzer crystals. Further, a second angle image of the object can be detected from a second diffracted beam emitted from analyzer crystals positioned at a second angular position. The second angle image of the object can be detected at a high rocking curve angle setting of the analyzer crystals. The first and second angle images can be combined by a computer to derive a refraction image and apparent absorption image. Further, the computer can derive a mass density image of the object from the refraction image. The mass density image can be presented to a user via a display of the computer.

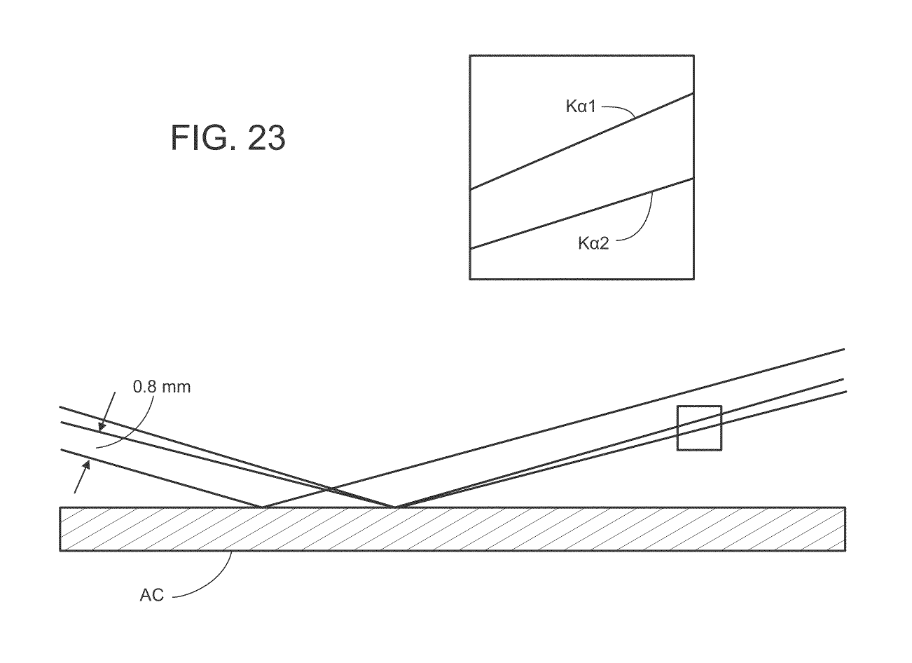

FIG. 23 is a side view of an analyzer crystal AC of any one of the DEI systems shown in FIGS. 9-19 and 21 according to an embodiment of the subject matter described herein. Referring to FIG. 23, the diffraction of characteristic emission lines Kα1 and Kα2 from the surface of analyzer crystal AC are shown. The accommodation of more than one x-ray energy can result in improved X-ray flux.

In another embodiment, a DEI system in accordance with the subject matter described herein can include a mismatch crystal design for rejecting particular X-rays emitted by an X-ray tube. In this design, the Kα2 emission line of the X-ray beam can be eliminated at the monochromator. A collimator can be positioned for blocking a portion of an X-ray beam that fall outside an angular acceptance window of a first set of monochromator crystals, such as, for example, one of monochromator crystals MC1-1-MC1-n shown in FIG. 13. The unblocked portion of the X-ray beam can intercept the first monochromator crystals, which refract the unblocked portions in a direction for intercept by a second set of monochromator crystals, such as, for example, one of monochromator crystals MC2-1-MC2-n shown in FIG. 13. The first set of monochromator crystals can be tuned to a particular angle using Bragg's Law to select a very narrow range of photon energies for resulting in diffracted monochromatic beams directed towards the second set of monochromator crystals. Because of the divergence of the X-ray beam from a source point, the first set of monochromator crystals can diffract a range of energies which can include the characteristic emission lines Kα1 and Kα2 and bremsstrahlung radiation at nearby energies. A function of the second set of monochromator crystals is to redirect the beam to a direction parallel to the incident beam and aligned with a set of analyzer crystals, such as, for example, analyzer crystals AC-1-AC-n shown in FIG. 13. When tuning the system for a particular energy, the monochromator crystals of the first set are aligned first, and then the monochromator crystals of the second set are tuned to find the position of the beam.

The monochromator crystals of the first and second sets can be configured in a mismatch crystal design for rejecting particular X-ray beams emitted by source points, such as small area sources of an X-ray tube. The monochromator crystals can be used to eliminate the Kα2 emission line of the X-ray beam, which can be achieved by utilizing the angular acceptance versus energy for different crystals. In one example, the monochromator crystals can be germanium [333] and silicon [333] monochromator crystals, respectively.

In another example of detecting the image of the object, first angle image of an object can be detected from first diffracted beams emitted from analyzer crystals positioned at first angular positions. The first angle image of an object can be detected at a low rocking curve angle setting of the analyzer crystals. Further, a second angle image of the object can be detected from second diffracted beams emitted from analyzer crystals positioned at second angular positions. The second angle images of the object can be detected at a high rocking curve angle setting of the analyzer crystals. The first and second angle images can be combined by a computer to derive a refraction image. Further, the computer can derive a mass density image of the object from the refraction image. The mass density image can be presented to a user via a display of the computer.

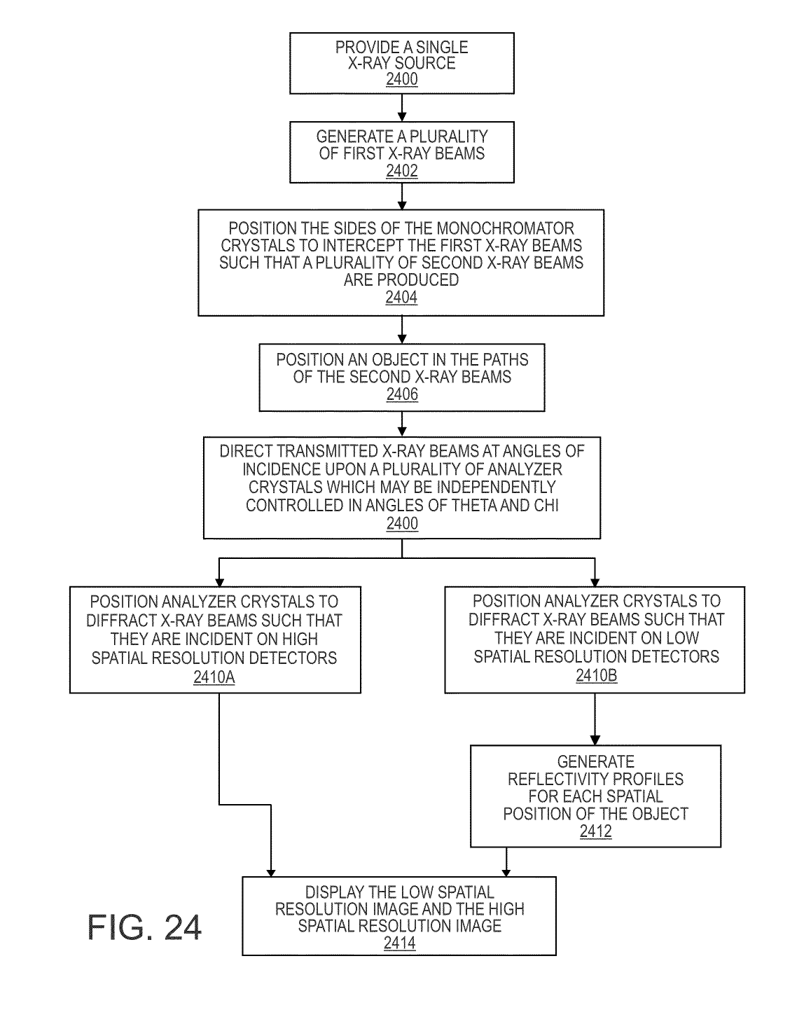

FIG. 24 is a flow chart illustrating an exemplary process for the simultaneous acquisition of high spatial resolution images and low spatial resolution, and high angular resolution images of an object by use of a DEI system, such as one of the DEI systems shown in FIGS. 13-19 and 21, according to an embodiment of the subject matter described herein. Referring to FIG. 24, in step 2400, a single X-ray source may be provided. In step 2402 the single X-ray source may generate a plurality of first X-ray beams. As an example, the X-ray beams may be generated by X-ray tube XT. Sides S1 of monochromator crystals MC-1-MC-n-1 may be positioned to intercept a respective one of the first X-ray beams such that a plurality of second X-ray beams each having predetermined energy levels is produced (step 2404). For example, a surface S1 of each of the monochromator crystals can be positioned in the path of its respective X-ray beam for intercepting the beam. Each monochromator crystal can be adapted to reject the majority of photons of its respective X-ray beam that does not have a desired energy. Thus, a resulting second X-ray beam XB2 can be produced that has the predetermined energy level. In one example, a surface of each monochromator crystal can be positioned at an angle of between about 5 degrees and 20 degrees with respect to a path of its respective X-ray beam incident upon the surface of the monochromator crystal. In this example, these angles may be used for [333] reflection. Alternatively, other suitable angles may be used in the positioning of the surface of the monochromator crystal. In another example, a surface of the monochromator crystal can be positioned at an angle of between about 1 degrees and 20 degrees with respect to a path of its respective X-ray beam incident upon the surface of the monochromator crystal. In another example, a surface of each monochromator crystal can be positioned at an angle of between about 1 degree and 20 degrees with respect to a path of its respective X-ray beam incident upon the surface of monochromator crystal MC. If the [555], [444], [333] and [111] reflections are used, the angular range can be between about 1 degree and about 40 degrees for the energy range of 10 to 100 keV.

In step 2406, an object O can be positioned in the paths of the second X-ray beams XB2 for transmission of the second X-ray beams XB2 through the object and emission from the object transmission X-ray beams.

In step 2408, the transmitted X-ray beams XB2 can be directed at angles of incidence upon the analyzer crystals AC1-AC-n generating third X-ray beams, XB3. Each analyzer crystal AC1-AC-n may be independently controlled in angles theta and chi. The analyzer crystals AC and third X-ray beams XB3 are divided into two groups: Analyzer crystals AC-H may be angled such that they diffract X-ray beams XB3H, these X-ray beams, XB3H are incident on high spatial resolution detectors DH; and Analyzer crystals ACL may be angled such that they diffract X-ray beams XB3L, these X-ray beams XB3L are incident on low spatial resolution detectors DL. In step 2410A, the analyzer crystals AC-H may be each rotated to the same angle theta TH prior to imaging. The analyzer crystals AC-H may each remain at the angle TH and an image of the object can be detected from the diffracted X-ray beams DXB-H. In step 2410B, the analyzer crystals AC-H may be each rotated to the same angle theta TH prior to imaging. The analyzer crystals AC-L may be cycled through a range of angles TL-1-TL-N, with respect to the Bragg diffraction peak, for each position as the object is scanned. A low spatial resolution image of the object can be detected from the diffracted X-ray beams DXB-L for each analyzer crystal angle TL-1-TL-N.

In step 2412, corresponding pixels from the series of images at analyzer crystal angles TL-1-TL-N can then be used to generate reflectivity profiles for each spatial position of the object. In one example, the reflectivity profile at each point could be fit to a Gaussian and a new image could be generated where each pixel represents the width of the Gaussian at each position in the object. In another example, the sum across all of the images TL-1-TL-N could be calculated for each pixel, and a new image can be generated that represents this sum over the reflectivity profile.

In step 2414, the generated low spatial resolution image(s) (2410B) from the reflectivity profile can be co-registered to the high spatial resolution image (2410A) and the images can be displayed together. In one example, the high spatial resolution image can be displayed in gray-scale and the low spatial resolution can be displayed in color with an opacity of less than 100%.

In another example of detecting the high spatial resolution image of the object, a first angle image of object can be detected from first diffracted beams emitted from analyzer crystals positioned at a first angular position. The first angle image of the object can be detected at a low rocking curve angle setting of the analyzer crystals. Further, a second angle image of the object can be detected from a second diffracted beam emitted from analyzer crystals positioned at a second angular position. The second angle image of the object can be detected at a high rocking curve angle setting of the analyzer crystals. The first and second angle images can be combined by a computer to derive a refraction image and apparent absorption image. Further, the computer can derive a mass density image of the object from the refraction image. The mass density image can be presented to a user via a display of the computer.

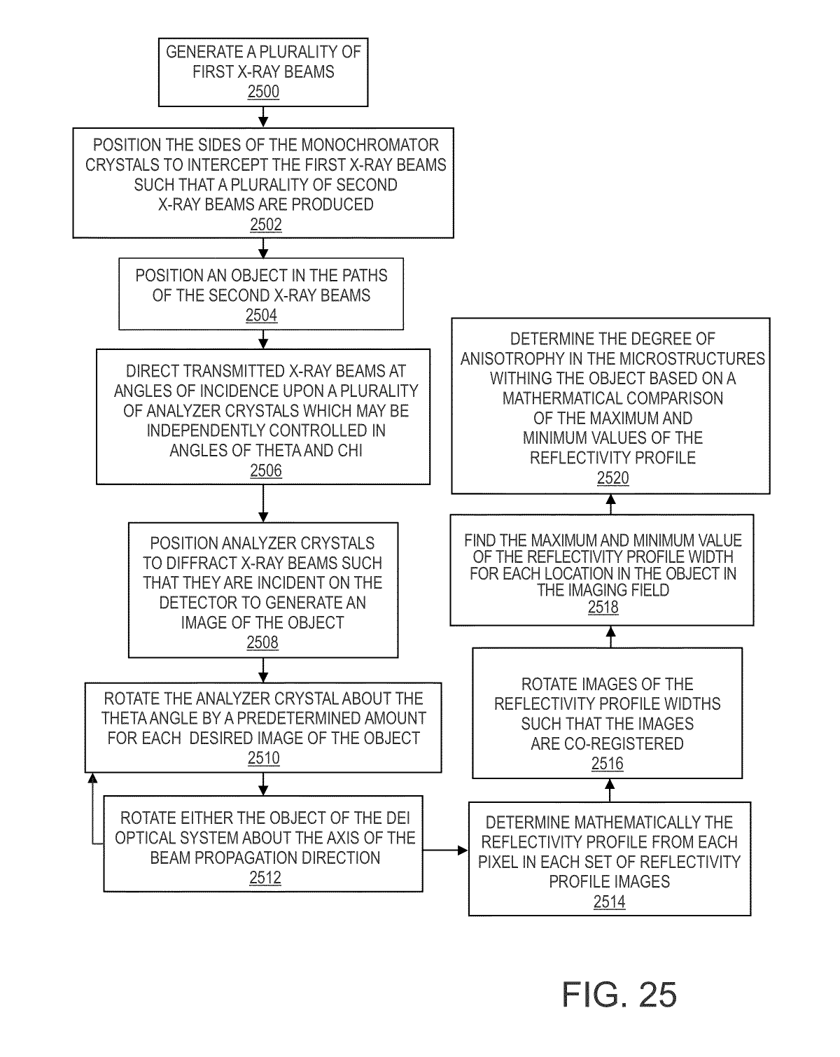

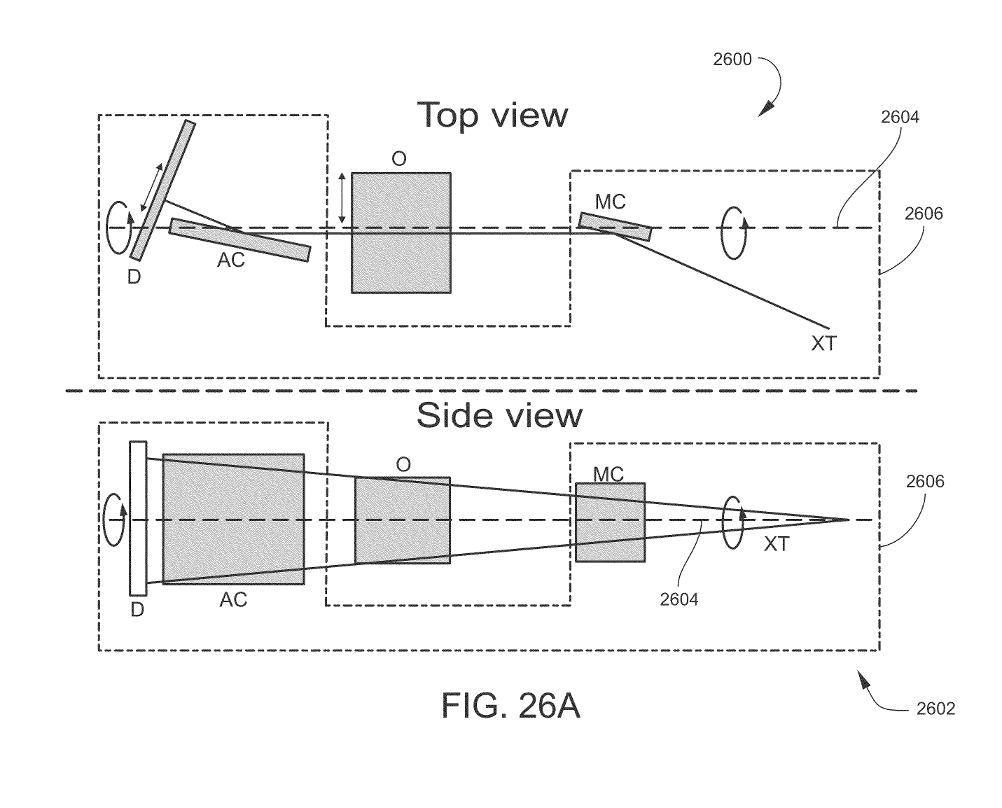

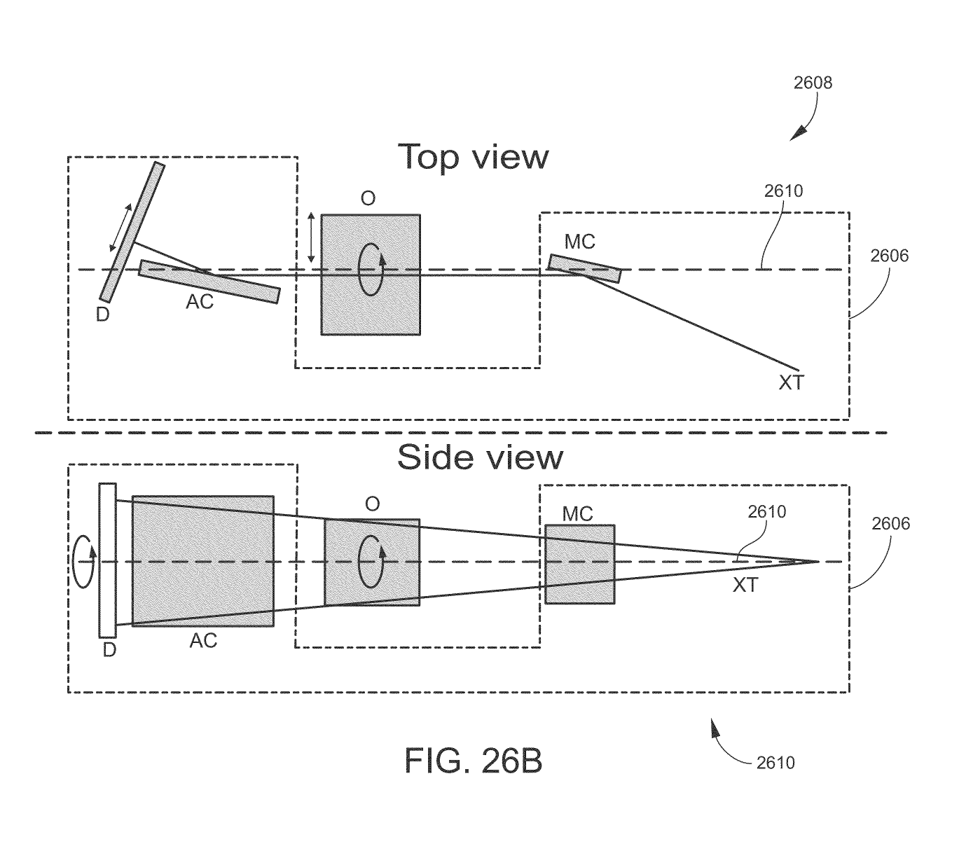

FIG. 25 is a flow chart illustrating an exemplary process for the acquisition of high angular resolution images at a range of object orientations with respect to a DEI optical system, such as one of the DEI systems shown in FIGS. 26A and 26B, according to an embodiment of the subject matter described herein. Referring to FIG. 25, in step 2500, a singular or plurality of first X-ray beams may be generated by X-ray tube XT. Sides S1 of monochromator crystal or crystals MC-1-MC-n-1 are positioned to intercept a respective one of the first X-ray beams such that a singular or plurality of second X-ray beams each having predetermined energy levels is produced (step 2502). For example, a surface S1 of each of the monochromator crystals can be positioned in the path of its respective X-ray beam for intercepting the beam. Each monochromator crystal can be adapted to reject the majority of photons of its respective X-ray beam that does not have a desired energy. Thus, a resulting second X-ray beam XB2 can be produced that has the predetermined energy level. In one example, a surface of each monochromator crystal can be positioned at an angle of between about 5 degrees and 20 degrees with respect to a path of its respective X-ray beam incident upon the surface of the monochromator crystal. In this example, these angles may be used for [333] reflection. Alternatively, other suitable angles may be used in the positioning of the surface of monochromator crystal. In another example, a surface of the monochromator crystal can be positioned at an angle of between about 1 degrees and 20 degrees with respect to a path of its respective X-ray beam incident upon the surface of the monochromator crystal. In another example, a surface of each monochromator crystal can be positioned at an angle of between about 1 degree and 20 degrees with respect to a path of its respective X-ray beam incident upon the surface of monochromator crystal MC. If the [555], [444], [333] and [111] reflections are used, the angular range can be between about 1 degree and about 40 degrees for the energy range of 10 to 100 keV.

In step 2504, an object O can be positioned in the paths of the second X-ray beams XB2 for transmission of the second X-ray beams XB2 through the object and emission from the object transmission X-ray beams.

In step 2506, the transmitted X-ray beams XB2 can be directed at angles of incidence upon the analyzer crystal or crystals AC1-AC-n generating third X-ray beams, XB3. Each analyzer crystal or crystals AC1-AC-n can be independently controlled in angles theta and chi.

In step 2508, for a single analyzer crystal angle, the third x-ray beam XB3 can be detected by an x-ray detector to generate an image of the object.

In step 2510, the analyzer crystal can be rotated about the theta angle by a predetermined amount (e.g., on the order of tenths of microradians to microradians), and another image of the object can be detected. This can be repeated at least twice until a set of reflectivity profile images is generated.

In step 2512, one of either the object or the DEI optical system is rotated about the axis of the beam-propagation direction (FIGS. 26A and 26B). Steps 2506 to 2510 are repeated to generate a second set of reflectivity profiles for the new object orientation relative to the x-ray optics.

In step 2514, the widths of the reflectivity profile from each pixel in each set of reflectivity profile images can be mathematically determined. In one example, the width is measured by fitting the reflectivity profile to a Gaussian. Once this step is completed, there will be an image of the reflectivity profile width of the object for each object orientation.

In step 2516, images of the reflectivity profile widths can be each rotated, such that the images are co-registered. These set of co-registered images then represent a map of the reflectivity profile width as a function of orientation angle of the object.

In step 2518, the maximum and minimum value of the reflectivity profile width can be found for each location in the object and in the imaging field. As an example, the system may use the plurality of intensity measurements to determine the maximum and minimum reflectivity profile widths.

In step 2520, the maximum and minimum values of the reflectivity profile can be mathematically compared to determine the degree of anisotropy in the microstructures within the object.

Referring to FIG. 26A, illustrate the Top View 2600 and Side View 2602 of the system and the central dotted line 2604 of the system's axis of rotation (SAR). In the case illustrated in FIG. 26A, the object may be held stationary in angle, a, while an imaging system 2606 including that includes the X-ray source (XT), monochromator crystal (MC), analyzer crystal (AC) and detector (D) can be rotated by some angle, α, about the depicted axis of rotation (SAR). FIG. 26B illustrates the top view 2608 and side view 2610 of the system and the central dotted line 2604 in both 2608 and 2610 illustrating the object's axis of rotation (OAR). In the case depicted in FIG. 26B, the system may be held at a constant angle, α, while the object is rotated by an angle, α, about the object's axis of rotation (OAR).

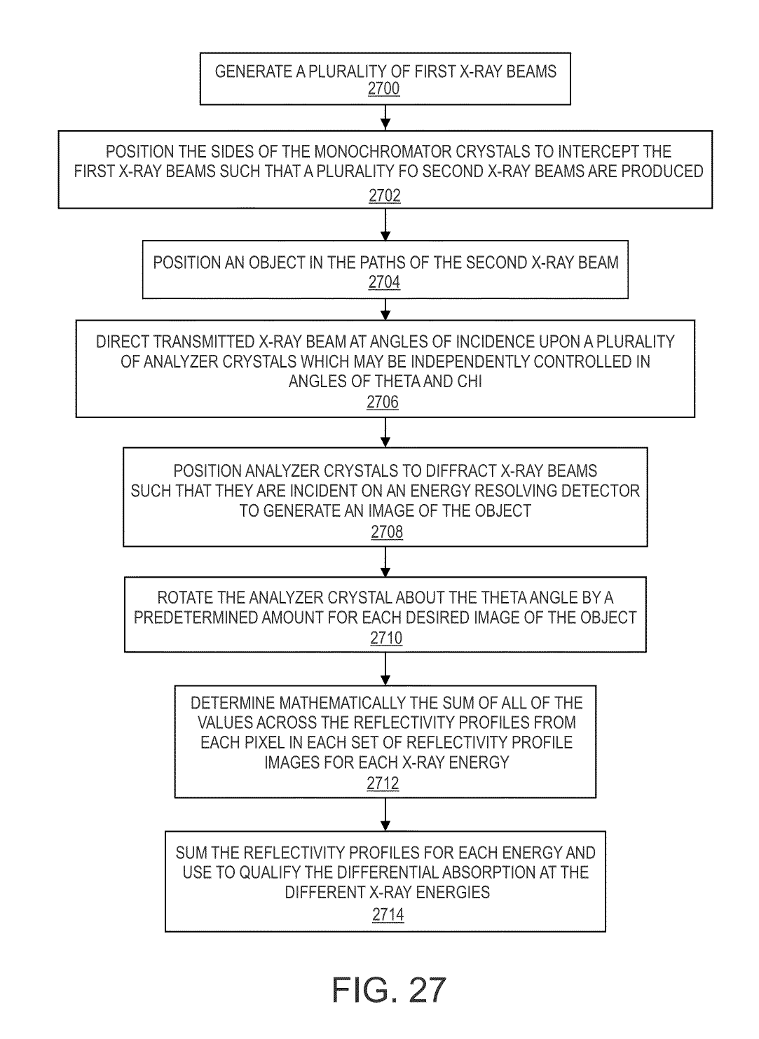

FIG. 27 is a flow chart illustrating an exemplary process for the simultaneous acquisition of high angular resolution images at multiple X-ray energies with a DEI optical system, such as one of the DEI systems shown in FIGS. 26A and 26B, according to an embodiment of the subject matter described herein. Referring to FIG. 27, in step 2700, a singular or plurality of first X-ray beams may be generated by X-ray tube XT. Sides S1 of monochromator crystal or crystals MC-1-MC-n-1 can be positioned to intercept a respective one of the first X-ray beams such that a singular or plurality of second X-ray beams each having predetermined energy levels can be produced (step 2702). For example, a surface S1 of each of the monochromator crystals can be positioned in the path of its respective X-ray beam for intercepting the beam. Each monochromator crystal can be adapted to reject the majority of photons of its respective X-ray beam that does not have a desired energy. Thus, a resulting second X-ray beam XB2 can be produced that has the two or more predetermined energy levels. In one example, a surface of each monochromator crystal can be positioned at an angle of between about 1 degrees and 20 degrees with respect to a path of its respective X-ray beam incident upon the surface of the monochromator crystal. In this example, these angles may be used for the [111], [333], [444] and [555] reflections. Alternatively, other suitable angles may be used in the positioning of the surface of monochromator crystal. In another example, the energy of the portions of the X-ray beam corresponding to the [111], [333], [444] and [555] reflections will have an energy range of about 10 to 100 keV.

In step 2704, an object O can be positioned in the paths of the second X-ray beams XB2 for transmission of the second X-ray beams XB2 through the object and emission from the object transmission X-ray beams.

In step 2706, the transmitted X-ray beams XB2 can be directed at angles of incidence upon the analyzer crystal or crystals AC1-AC-n generating third X-ray beams, XB3. Each analyzer crystal or crystals AC1-AC-n be independently controlled in angles theta and chi.

In step 2708, for a single analyzer crystal angle, the third x-ray beam XB3 can be detected by an energy resolving x-ray detector to generate an image of the object. In one example, separate images are detected for each of the X-ray energies corresponding to the [333], [444] and [555] reflections.

In step 2710, the analyzer crystal is rotated about the theta angle by a predetermined amount (e.g., on the order of tenths of microradians to microradians), and another set of energy-resolved images of the object are detected. In one example, a set of reflectivity profile images can be generated for each of the X-ray energies corresponding to the [333], [444], and [555] reflections.

In step 2712, the sum of all of the values across the reflectivity profiles and the widths of the reflectivity profiles from each pixel in each set of reflectivity profile images for each X-ray energy can be mathematically determined. Once this step can be completed, there may be an image of the sum over the reflectivity profile for each energy of the X-ray beam and an image of the reflectivity profile width for each energy of the X-ray beam. In one example, the sum over the reflectivity profiles and reflectivity profiles widths for the X-ray energies corresponding to the [333], [444] and [555] reflections may be separately determined.

In step 2714, the sum over the reflectivity profiles for each energy may be used to quantify the differential absorption at the different X-ray energies through dual-energy X-ray absorptiometry. In one example, the amount of mineral content in the object can be quantified.

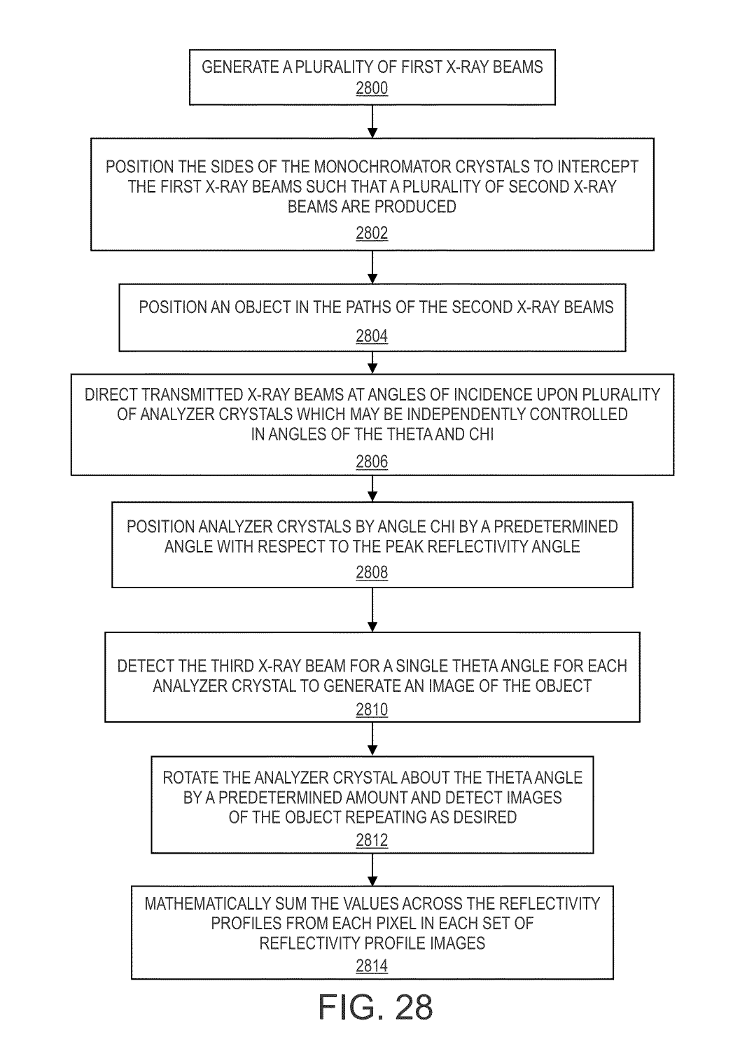

FIG. 28 is a flow chart illustrating an exemplary process for the acquisition of high angular resolution images at multiple X-ray energies with a DEI optical system with the chi angle of the analyzer crystals shifted by a predetermined amount from the aligned angular position, such as one of the DEI systems shown in FIGS. 3, 6 and 7, according to an embodiment of the subject matter described herein. Referring to FIG. 28, in step 2800, a singular or plurality of first X-ray beams may be generated by X-ray tube XT. Sides S1 of monochromator crystal or crystals MC-1-MC-n-1 are positioned to intercept a respective one of the first X-ray beams such that a singular or plurality of second X-ray beams each having predetermined energy levels can be produced (step 2802). For example, a surface S1 of each of the monochromator crystals can be positioned in the path of its respective X-ray beam for intercepting the beam. Each monochromator crystal can be adapted to reject the majority of photons of its respective X-ray beam that does not have a desired energy. Thus, a resulting second X-ray beam XB2 can be produced that has the two or more predetermined energy levels. In one example, a surface of each monochromator crystal can be positioned at an angle of between about 1 degrees and 20 degrees with respect to a path of its respective X-ray beam incident upon the surface of the monochromator crystal. In this example, these angles may be used for the [111], [333], [444] and [555] reflections. Alternatively, other suitable angles may be used in the positioning of the surface of monochromator crystal. In another example, the energy of the portions of the X-ray beam corresponding to the [111], [333], [444] and [555] reflections will have an energy range of about 10 to 100 keV.

In step 2804, an object O can be positioned in the paths of the second X-ray beams XB2 for transmission of the second X-ray beams XB2 through the object and emission from the object transmission X-ray beams.

In step 2806, the transmitted X-ray beams XB2 can be directed at angles of incidence upon the analyzer crystal or crystals AC1-AC-n generating third X-ray beams, XB3. Each analyzer crystal or crystals AC1-AC-n can be independently controlled in angles theta and chi.

In step 2808, the analyzer crystal or crystals AC1-AC-n may be tilted in angle chi by a predetermined angle with respect to the peak reflectivity angle (in chi).

In step 2810, for a single theta angle for each analyzer crystal, the third x-ray beam XB3 can be detected by an x-ray detector to generate an image of the object.

In step 2812, the analyzer crystal can be rotated about the theta angle by a predetermined amount (typically on the order of tenths of microradians to microradians), and detect images of the object. This can be repeated at least twice until sets of reflectivity profile images are generated for the X-ray beam.

In step 2814, the sum of all of the values across the reflectivity profiles and the widths of the reflectivity profiles from each pixel in each set of reflectivity profile images can be mathematically determined. In one example, the width is measured by fitting the reflectivity profile to a Gaussian. Once this step is completed, there will be an image of the sum over the reflectivity profile of the X-ray beam and an image of the reflectivity profile width of the X-ray beam. In another example, steps 2806 to 2814 may be executed with no object in the X-ray beam in order to determine the theta angle of the peak reflectivity as a function of position in the chi-angle-detuned X-ray beam. With this map of relative theta-angle-variations across the object-free X-ray beam, the individual pixels in the reflectivity profile images of the object can then be remapped to the corresponding theta-angle position.

In accordance with embodiments, the subject matter described herein can include a method for acquiring high speed and/or high angular resolution DEI reflectivity profiles. In DEI, the diffraction properties of perfect silicon crystals can be exploited to convert small deviations in the angle of the X-ray beam (on the order of tenths of microradians) caused by refraction from structures along the X-ray beam path into intensity differences in an image. A DEI system has at minimum 1 monochromator crystal, which monochromates and collimates the X-ray beam, and at minimum 1 analyzer crystal. The X-ray beam at the sample/patient location is a slot X-ray beam. Typically, prior to obtaining a DEI image, the diffraction planes of the monochromator and analyzer crystals are made parallel within about 0.1 microradians. This alignment may require that both the analyzer crystal's theta- and chi-angles to be aligned (angles defined in FIG. 3). Once this initial alignment is obtained, images can be taken with the analyzer crystal at a range of n-positions about the reflectivity peak (parallel to the monochromator) position to generate a reflectivity profile (x-ray intensity as a function of chi-angle about the parallel, peak, point) for the X-ray optics system. According to another aspect, the subject matter described herein can include a system and method for the simultaneous acquisition of a high spatial resolution single DEI image and a series of low spatial resolution DEI reflectivity profile images. Once this initial alignment is obtained, one of two different types of images or image sets may be obtained. In the first type, DEI radiographs may be obtained by rotating the analyzer crystal to the preferred angular position, then fixing the analyzer crystal in place while the slot X-ray beam is scanned across the full imaging field. In a second type, multiple DEI images may be acquired for a range of analyzer crystal angles.

In accordance with the present disclosure, the DEI system can be configured such that a DEI optical system or an array of DEI optical systems will intercept the divergent X-ray beam from the X-ray source. The beam emerging from each individual optics systems within the array can then be incident upon either a high-spatial resolution detector or an energy-resolving, low-spatial resolution (˜1 mm̂2 pixels) detector. The DEI optical systems for both the high-spatial resolution and low-spatial resolution portions of the X-ray beam can be operated independently of one another; the angular position of the analyzer crystal for the high-spatial resolution portion of the beam is independent of the analyzer crystal position for the low-spatial resolution portion of the beam. For the beam that is incident upon the high-spatial resolution detector, the small pixel size of the detector can allow for detailed imaging of anatomical information or of structural information for structures of about 50 microns or larger. For the portion of the X-ray beam that is incident on the low-spatial resolution detector, the large pixel area and high-efficiency of the photon counting detector can allow for the high-speed measurement of the reflectivity profile, which can give detailed information about microstructural features (features smaller than about 100 microns) in the imaging field. The data from the two types of images can then be superimposed to combine structural/anatomical and microstructural information about the imaging object.

The reflectivity profile may have clinical utility because the width of the reflectivity profile is proportional to the number of refracting microstructures along the X-ray beam path. Simultaneous acquisition of high-spatial resolution structural/anatomical information and microstructural information can greatly improve clinicians' ability to diagnose and localize injuries or disease, especially those in the lung or bone. Beyond clinical utility of this approach, this could be used for industrial inspection. Any scenario in which it's advantageous to simultaneously reveal structural and microstructure information could benefit greatly.

According to another aspect, the subject matter described herein can include a system comprising an X-ray generation device configured to generate a plurality of first X-ray beams. The system can include monochromator crystals including first and second sides. The first sides of the monochromator crystals are positioned in predetermined positions to directly intercept the plurality of first X-ray beams for generating a plurality of second X-ray beams. The second sides of the monochromator crystals are positioned to intercept the plurality of second X-ray beams such that a plurality of third X-ray beams is produced for transmission through an object. A plurality of analyzer crystals are positioned to intercept transmitted X-ray beams at angles of incidence of the analyzer crystals. An image detector can be configured to detect an image of the object from beams diffracted from the analyzer crystals. An image of the object may be detected based upon beams diffracted from the analyzer crystals. These systems and methods can be advantageous, for example, because they can provide extremely low dose in medical applications, fast scan times, high resolution, and relatively low operation and build costs. Further, for example, these systems can be constructed into a compact unit and be readily usable in clinical and industrial applications. Additional description about these systems and related methods are described in further detail herein.

An image processing technique using DEI in accordance with the subject matter described herein can use images acquired at symmetric points of the rocking curve to generate apparent absorption and refraction images of an object. A DEI apparent absorption image is similar to a conventional radiograph image, but exhibits much greater contrast owing to scatter rejection. DEI refraction images can depict the magnitude of small beam deflections caused by large-scale refractive-index features (features of a size at or greater than the system resolution). A DEI extinction image is generated at points on the rocking curve where the primary mechanism of contrast is due to photons that have been scattered by an object on the order of microradians. Another DEI based imaging processing technique may be referred to as Multiple Image Radiography (MIR) which uses multiple points on the rocking curve to generate quantitative images representing an object's X-ray absorption, refraction, and ultra-small angle scatter. Systems and methods can generate images at any point on the analyzer rocking curve, and can thus be used to generate: (1) single image DEI at any analyzer position; (2) DEI apparent absorption and refraction images; and (3) mass density images. The ability to generate the raw image data required for these processes and any other DEI based processing technique are useful for all DEI based processing techniques. In addition, systems and methods described herein are amenable for use in computed tomography, and can provide the raw data for use in any DEI-based computed tomography algorithm.

As understood, a small area source may refer to any source capable of generating X-ray beams from a small area in space. For example, an X-ray tube may include multiple small area sources for emitting X-ray beams from multiple points. The small area sources may be within the same X-ray tube source. Alternatively or in addition to being a part of a system as disclosed herein, multiple X-ray tube sources may each provide one or more small area sources and be used together for generating multiple X-ray beams.

Approaches to DEI or analyzer-based imaging as described herein can use large X-ray beams at a sample or object location to image the object without the need to scan the X-ray beam. These large area X-ray beams can be generated through the use of asymmetric crystals, an X-ray line source, or a combination of the two. As with the techniques and systems presented herein, the other techniques may require a high-power X-ray tube source operating at a peak voltage well above the Kα1 emission energy of their respective source in order to generate sufficient Kα1 flux for a small imaging time. The high energy X-rays generated by the high peak voltage will be readily scattered by the monochromator crystals, and this scattered radiation dose delivered to the object to be imaged. Stated in another way, there will be a “line-of-sight” between the scatter locations on the monochromator crystals and the object to be imaged over which there cannot be significant radiation shielding to stop the scattered radiation from reaching the object to be imaged. This contribution of scattered radiation to the radiation dose delivered to the object to be imaged can be overcome through the use of a multiple small-vertical height X-ray beam system, which can be created through the use of an array of small area X-ray beams. Any radiation that does not propagate along the narrow beam path can be filtered out by high-Z shielding, and therefore only a minimal amount of scattered radiation will reach the object to be imaged.

### Exemplary Applications

The systems and methods in accordance with the subject matter described herein can be applied to a variety of medical applications. As set forth above, the systems and methods described herein can be applied for breast imaging. Further, for example, the systems and methods described herein can be applied to cartilage imaging, neuroimaging, cardiac imaging, vascular imaging (with and without contrast), pulmonary (lung) imaging, bone imaging, genitourinary imaging, gastrointestinal imaging, soft tissue imaging in general, hematopoietic system imaging, and endocrine system imaging. In addition to image time and dose, a major advancement of using higher energy X-rays may be the thickness of the object that can be imaged. For applications such as breast imaging, the system described allows for imaging full thickness breast tissue with a clinically realistic imaging time. The same can be said for other regions of the body, such as the head, neck, extremities, abdomen, and pelvis. Without the limitations of X-ray absorption, utilization of DEI with higher energy X-rays dramatically increases the penetration ability of X-rays. For soft tissue, only a small portion of the X-ray photons incident on the object may be absorbed, which greatly increases efficiency of emitted photons from the X-ray tube reaching the detector.

With respect to pulmonary imaging, DEI techniques as described herein can produce excellent contrast in the lungs and can be used heavily for diagnosing pulmonary conditions such as pneumonia. Fluid collections in the lungs generate a marked density gradient that could be detected easily with DEI. The density gradient, characteristics of the surrounding tissue, and geometric differences between normal lung tissue and tissue with a tumor can be large, producing good contrast. Further, DEI techniques described herein can be applied to lung cancer screening and diagnosis. The high angular resolutions of the reflectivity profile may reveal microstructural information about the lungs including the degree to which the alveoli are aerated.

With respect to bone imaging, DEI techniques as described herein can produce an excellent image of bone in general. For the high spatial resolution structural imaging, the high refraction and extinction contrast of DEI can be especially useful for visualizing fractures and lesions within the bone. The high angular resolution images from the reflectivity profile reveals properties of the bone microstructure that can be indicative of microarchitectural deterioration of the bone.

Further, the systems and methods in accordance with the subject matter described herein can be applied to a variety of inspection and industrial applications. For example, the systems and methods can be applied for meat inspection, such as poultry inspection. For example, the systems and methods can be used for viewing sharp bones, feathers, and other low contrast objects in meats that required screening and/or removal. The systems and methods described herein can be applied for such screening.

The systems and methods described herein can also be applied for manufacture inspection. For example, the systems and methods can be used for inspecting welds, such as in aircraft production. DEI techniques as described herein can be used to inspect key structural parts that undergo heavy wear and tear, such as jet turbine blades. Further, for example, the systems and methods described herein can be used for inspecting circuit boards and other electronics. In another example, the systems and methods described herein can be used for tire inspection, such as the inspection of steel belts and tread integrity.

Further, the systems and methods in accordance with the subject matter described herein can be used for security screening purposes. For example, the systems and methods can be used for screening at airports and seaports. DEI techniques as described herein can be used for screening for plastic and low absorption contrast objects, such as plastic knives, composite guns difficult to detect with conventional X-ray, and plastic explosives. For imaging larger objects, such as for airport baggage inspection, the distance between the X-ray tube and detector can be increased to allow beam divergence. A larger analyzer crystal may be necessary to accommodate a larger fan beam.

The device described provides a mechanism that can be translated into a computed tomography imaging system, or DEI-CT. A DEI-CT system, resembling a third generation conventional computed tomography system, may use the same apparatus but modified for rotation around a central point. Alternatively, the system could remain stationary and the object, sample, or patient could be rotated in the beam. A DEI-CT system of this design would produce images representing X-ray absorption, refraction, and ultra-small angle scatter rejection (extinction), but they would be resolved in three dimensions.

The various techniques described herein may be implemented with hardware or software or, where appropriate, with a combination of both. Thus, the methods and apparatus of the disclosed embodiments, or certain aspects or portions thereof, may take the form of program code (i.e., instructions) embodied in tangible media, such as floppy diskettes, CD-ROMs, hard drives, or any other machine-readable storage medium, wherein, when the program code is loaded into and executed by a machine, such as a computer, the machine becomes an apparatus for practicing the subject matter disclosed herein. In the case of program code execution on programmable computers, the computer will generally include a processor, a storage medium readable by the processor (including volatile and non-volatile memory and/or storage elements), at least one input device and at least one output device. One or more programs are preferably implemented in a high level procedural or object oriented programming language to communicate with a computer system. However, the program(s) can be implemented in assembly or machine language, if desired. In any case, the language may be a compiled or interpreted language, and combined with hardware implementations.

The described methods and apparatus may also be embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, a video recorder or the like, the machine becomes an apparatus for practicing the subject matter disclosed herein. When implemented on a general-purpose processor, the program code combines with the processor to provide a unique apparatus that operates to perform the processing of the presently disclosed subject matter.

While the embodiments have been described in connection with the preferred embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed:

1. A method for detecting an image of an object, the method comprising:
providing a single X-ray source;
generating a first X-ray beam using the single X-ray source;
positioning a plurality of monochromator crystals to intercept the first X-ray beam such that a plurality of second X-ray beams each having predetermined energy level, is produced;
positioning an object in paths of the plurality of second X-ray beams for transmission of the plurality of second X-ray beams through the object and emitting from the object a plurality of transmitted X-ray beams;
directing the plurality of transmitted X-ray beams at angles of incidence upon a plurality of analyzer crystals, wherein the angles of incidence of the analyzer crystals are independently adjustable; and
detecting an image of the object from each of the X-ray beams diffracted from each analyzer crystal using a plurality of detectors.

2. The method of claim 1, wherein the plurality of detectors comprise at least one high spatial resolution detector and at least one low spatial resolution detector.

3. The method of claim 2, wherein the low spatial resolution detector is an energy-resolving detector.

4. The method of claim 1, further comprising:
adjusting the angles of incidence of the analyzer crystals, and
detecting a plurality of images of the object during adjustment of the angles of incidence of the analyzer crystals.

5. The method of claim 4, wherein directing the plurality of transmitted X-ray beams comprises directing the rotation of the analyzer crystals about a propagation direction of the transmitted X-ray beams relative to the analyzer crystal.

6. The method of claim 4, wherein detecting an image of the object comprises tilting the analyzer crystal out of alignment by a predetermined chi-angle; and
wherein the method further comprises detecting a plurality of images of the object in sequence for a range of theta-angular positions of the analyzer crystals.

7. The method of claim 4, further comprising using the detectors to measure the intensity of the diffracted X-ray beam.

8. The method of claim 7, further comprising using the measured intensity of the diffracted X-ray beam to determine the degree of anisotropy in a structure of the object.

9. The method of claim 7, further comprising using the measured intensity of the diffracted X-ray beam to determine the orientation direction of structures in the object.

10. The method of claim 7, wherein measuring the intensity of the diffracted X-ray beam comprises detecting a plurality of intensity measurements for a range of angular positions of the analyzer crystal.

11. The method of claim 10, wherein the range of angular positions is a range of angles the X-ray source is rotated about the propagation direction of the X-ray beam relative to the analyzer crystals.

12. The method of claim 10, wherein the range of angular positions is a range of angles the object is rotated about the propagation direction of the X-ray beam relative to the analyzer crystals.

13. The method of claim 10, further comprising using the series of intensity measurements to determine the degree of anisotropy in the structure of the object.

14. The method of claim 10, further comprising using the series of intensity measurements to determine the orientation direction of the structures in the object.

15. The method of claim 10, wherein measuring the intensity of the diffracted X-ray beam comprises tilting the crystal analyzer out of alignment by a predetermined angle; and
wherein the method further comprises detecting a series of intensity measurements are obtained for a range of angular positions of the analyzer crystal.

16. The method of claim 10, further comprising using the plurality of intensity measurements to determine the maximum and minimum reflectivity profile widths.

17. A system for detecting an image of an object, the system comprising:
a single X-ray source configured to generate a first X-ray beam;
a plurality of monochromator crystals positioned to intercept the first X-ray beam such that a plurality of second X-ray beams each having predetermined energy level, is produced;
a plurality of analyzer crystals positioned to intercept a plurality of transmitted X-ray beams at an angle of incidence from the object, wherein the plurality of transmitted X-ray beams are emitted from the object positioned in the path of the plurality of second X-ray beams, and wherein the angles of incidence of the analyzer crystals are independently adjustable; and
a plurality of detectors configured to detecting an image of the object from each of the transmitted X-ray beams diffracted from each analyzer crystal.

18. The system of claim 15, wherein the plurality of detectors comprise at least one high spatial resolution detector and at least one low spatial resolution detector.

19. The method of claim 18, wherein the low spatial resolution detector is an energy-resolving detector.

20. The system of claim 17, further comprising:
the plurality of detectors configured to adjust the angles of incidence of the analyzer crystals, and
detect a plurality of images of the object during adjustment of the angles of incidence of the analyzer crystals.
